SEQUENCE LISTING (28 sequences)

SEQ ID NO: 1 - RNA, 41 nt, Synthetic construct
  source 1..41: mol_type = other RNA
taatttctactaagtgtagatagaggatacttcgtcgctgc

SEQ ID NO: 2 - DNA, 27 bp, Synthetic construct
  source 1..27: mol_type = other DNA
aagtatcctctgctgaaattattaggc

SEQ ID NO: 3 - DNA, 28 bp, Synthetic construct
  source 1..28: mol_type = other DNA
ctattacagttaatccgtgctttgtgtg

SEQ ID NO: 4 - DNA, 47 bp, Synthetic construct
  source 1..47: mol_type = other DNA
atttcgagatggtgagaaataagtatcctctgctgaaattattaggc

SEQ ID NO: 5 - DNA, 48 bp, Synthetic construct
  source 1..48: mol_type = other DNA
atttcgagatggtgagaaatctattacagttaatccgtgctttgtgtg

SEQ ID NO: 6 - DNA, 61 bp, Synthetic construct
  source 1..61: mol_type = other DNA
gaaaagagaaggtgtgagggtgggagatcttttcaagtatcctctgctgaaattattaggc

SEQ ID NO: 7 - DNA, 62 bp, Synthetic construct
  source 1..62: mol_type = other DNA
gaaaagagaaggtgtgagggtgggagatcttttcctattacagttaatccgtgctttgtgtg

SEQ ID NO: 8 - DNA, 61 bp, Synthetic construct
  source 1..61: mol_type = other DNA
agatttcgaaggtgtgagggtgggagagaaatctaagtatcctctgctgaaattattaggc

SEQ ID NO: 9 - DNA, 62 bp, Synthetic construct
  source 1..62: mol_type = other DNA
agatttcgaaggtgtgagggtgggagagaaatctctattacagttaatccgtgctttgtgtg

SEQ ID NO: 10 - DNA, 288 bp, Human papillomavirus
  source 1..288: mol_type = genomic DNA
tattatgtcctacatctgtgtttagcagcgacgaagtatcctctgctgaaattattaggcagcacttggccaaccactccgccgcgacccatcccaaagccgtcgccttgggcaccaaagaatcacagacgactatccagcgaccaagatcagagccagacaccggaaacccctgccacaccaataagttgttgcacagagactcagtggacagtgctccaattctcactgcagttaacagctcacacaaagcacggattaactgtaatagtaacactacacccatag

SEQ ID NO: 11 - RNA, 41 nt, Synthetic construct
  source 1..41: mol_type = other RNA
taatttctactaagtgtagatattttaccgtcaccaccacg

SEQ ID NO: 12 - DNA, 28 bp, Synthetic construct
  source 1..28: mol_type = other DNA
ccaaggtttacccaataatactgcgtct

SEQ ID NO: 13 - DNA, 27 bp, Synthetic construct
  source 1..27: mol_type = other DNA
tctttcattttaccgtcaccaccacga

SEQ ID NO: 14 - DNA, 62 bp, Synthetic construct
  source 1..62: mol_type = other DNA
agatttcgaaggtgtgagggtgggagagaaatctccaaggtttacccaataatactgcgtct

SEQ ID NO: 15 - DNA, 62 bp, Synthetic construct
  source 1..62: mol_type = other DNA
agatttcgaaggtgtgagggtgggagagaaatctatcttggactgagatctttcattttacc

SEQ ID NO: 16 - RNA, 1261 nt, Severe Acute Respiratory Syndrome coronavirus 2
  source 1..1261: mol_type = genomic RNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgatgggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 17 - DNA, 30 bp, Synthetic construct
  source 1..30: mol_type = other DNA
accgaggtcgaaacgtacgttctttctatc

SEQ ID NO: 18 - DNA, 30 bp, Synthetic construct
  source 1..30: mol_type = other DNA
ctacgctgcagtcctcgctcactgggcacg

SEQ ID NO: 19 - DNA, 64 bp, Synthetic construct
  source 1..64: mol_type = other DNA
agatttcgaaggtgtgagggtgggagagaaatctaccgaggtcgaaacgtacgttctttctatc

SEQ ID NO: 20 - DNA, 64 bp, Synthetic construct
  source 1..64: mol_type = other DNA
agatttcgaaggtgtgagggtgggagagaaatctctacgctgcagtcctcgctcactgggcacg

SEQ ID NO: 21 - RNA, 41 nt, Synthetic construct
  source 1..41: mol_type = other RNA
taatttctactaagtgtagatagggggcctgacggtatgat

SEQ ID NO: 22 - DNA, 27 bp, Synthetic construct
  source 1..27: mol_type = other DNA
atacactattcaacgtagtacaggaga

SEQ ID NO: 23 - DNA, 29 bp, Synthetic construct
  source 1..29: mol_type = other DNA
tgaatttatgatttgcatcttcagtgatt

SEQ ID NO: 24 - DNA, 61 bp, Synthetic construct
  source 1..61: mol_type = other DNA
agatttcgaaggtgtgagggtgggagagaaatctatacactattcaacgtagtacaggaga

SEQ ID NO: 25 - DNA, 63 bp, Synthetic construct
  source 1..63: mol_type = other DNA
agatttcgaaggtgtgagggtgggagagaaatcttgaatttatgatttgcatcttcagtgatt

SEQ ID NO: 26 - RNA, 41 nt, Synthetic construct
  source 1..41: mol_type = other RNA
taatttctactaagtgtagatggagtgtcaatattatctcc

SEQ ID NO: 27-28 - skipped (2 entries)

(19) United States
(12) Patent Application Publication
(10) Pub. No.: US 2026/0092336 A1
(43) Pub. Date: Apr. 2, 2026

(54) AMPLICON-DEPLETED CRISPR/CAS-REGULATED ISOTHERMAL MOLECULAR ASSAY AT SKIN TEMPERATURE ENABLING MULTI-LEVEL, EQUIPMENT-FREE, HOME-BASED SELF-TESTING OF VIRAL INFECTIONS

(71) Applicant: The Hong Kong University of Science and Technology, Hong Kong (CN)

(72) Inventors: I-Ming HSING, Hong Kong (CN); Yumeng CAO, Hong Kong (CN)

(21) Appl. No.: 18/902,014

(22) Filed: Sep. 30, 2024

(51) Int. Cl. C12Q 1/70; C12N 9/22; C12N 15/11; C12Q 1/6818; G01N 21/64; G01N 21/77; G01N 21/78

(52) U.S. Cl. CPC C12Q 1/701; C12N 9/22; C12N 15/11; C12Q 1/6818; G01N 21/6428; G01N 21/78; C12N 2310/20; C12N 2320/10; G01N 2021/6432; G01N 2021/6439; G01N 2021/7759

(57) ABSTRACT

The subject invention relates to a skin-temperature operated iNAAT for at-home testing, called CRISPR-regulated one-pot Recombinase Polymerase Loop-mediated Amplification (CoRPLA) with rationally designed loop primers that enhance isothermal amplification, specifically trigger Cas12 cis-cleavage for in-situ amplicon depletion and initiate concurrent Cas12 trans-cleavage-based signal amplification, to eliminate amplicon-related cross-contamination and enables semi-quantitative response to the initial target amount with sub-attomolar sensitivity (0.5 cps/μL).

### SEQUENCE LISTING

The Sequence Listing for this application is labeled “HKUS-199-SeqList-30Sep24.xml” which was created on Sep. 30, 2024 and is 26,218 bytes. The entire contents of the sequence listing is incorporated herein by reference in its entirety.

### FIELD OF THE INVENTION

The present invention is in the field of nucleic acid diagnostics, in particular the skin-temperature amplicon-depleted CRISPR-based point-of-care testing diagnostic assay. This invention involves using CRISPR/Cas12a enzyme to specifically recognize amplified nucleic acid products and destroy one of the primer binding regions in the product sequence through the rational design of the primer sequence for LbCas12a recognition by sgRNA so that it can eliminate or reduce false positive results in, for example, a non-laboratory setting.

### BACKGROUND OF THE INVENTION

In recent decades, numerous large-scale viral outbreaks have resulted in millions of deaths and significant economic losses¹⁻³. The COVID-19 pandemic has clearly demonstrated the importance of decentralized at-home testing for mitigating and controlling emerging infectious diseases¹. The most widely used at-home test is the rapid antigen test (RAT), but it suffers from low sensitivity and specificity⁴, and a time-consuming development process⁵⁻⁷. Conversely, nucleic acid amplification tests (NAATs) are highly sensitive and quickly adaptable to novel pathogens⁸, but they are prone to amplicon-derived contamination, leading to false positive results and requiring costly equipment to circumvent this issue^{9,10}. An ideal diagnostic assay would combine the speed and ease of use of RATs¹¹ with the sensitivity, specificity, and adaptability of NAATs¹¹.

CRISPR/Cas-based isothermal NAATs (iNAATs) have shown promise in this regard, offering higher sensitivity and specificity than traditional RATs¹²⁻¹⁵. Additionally, CRISPR/Cas platforms retain the programmable capability of NAATs, allowing for the rapid development of diagnostic kits at the onset of novel infectious diseases (NID)¹⁶. However, applying these techniques to home-based testing remains hindered by concerns of amplicon aerosol contamination causative of false positives, the perceived complexity of testing devices, long assay times, and limitations in quantitatively assessing viral loads. Amplicon aerosol contamination, arising from the uncontrolled amplification process in iNAATs, can lead to false positives in repeated tests^{14,17-19}. While closed systems and one-pot reactions have been developed to mitigate this risk, challenges remain. Amplification-free CRISPR-based cascades offer potential solutions without requiring additional equipment, but suffer from high background signal leakage due to target-independent signal transduction Recent amplification-based strategies adjusting the reaction kinetics via suboptimal PAM sequences aimed at reducing cis cleavage of amplicons at the initial stage of the amplification often compromise the signal transduction efficiency and still face issues with amplicon accumulation²²⁻²⁴. Therefore, the optimal CRISPR-based one-pot amplification reaction must maintain high target amplification efficiency for quick initiation and high signal transduction efficiency while completely depleting amplicons. A recent amplicon-depletion approach has shown that this is an effective way to eliminate the risk of aerosol contamination without impacting the performance of the reaction²³.

### BRIEF SUMMARY OF THE INVENTION

The subject invention pertains to novel methods for a loop-mediated isothermal amplification (RPLA)-like recombinase polymerase amplification system that quickly creates loop-containing amplicons in the initiation stage, triggered by a rationally designed bubble-primer. This design improves the amplification efficiency and achieves fast DNA amplification kinetics at 30° C. In embodiments, the amplification system of the subject invention is combined with an optimized CRISPR/Cas12 sgRNA, targeting the primer binding region to regulate the amplicon generation and deplete it afterward. This novel system, named CRISPR-regulated one-pot Recombinase Polymerase Loop-mediated Amplification (CoRPLA) comprises the PAM sequence within the bubble primer, further enhancing the programmability of CRISPR/Cas-based NAAT by eliminating sequence constraints on the target. In embodiments, the CoRPLA products are free from amplifiable strands due to the amplicon-depleting design, effectively eliminating amplicon aerosol-derived false positives. Moreover, the signal generation is dominated by the Cas12 trans-cleavage with linear kinetics, facilitating semi-quantitative end-point signals. To further demonstrate the advantages of contamination-free and a semi-quantitative response, we enhanced the practicality of CoRPLA for home-based testing by integrating it into a skin-adherent test tape called CoRPLA-tape. In this wearable design, the CoRPLA reaction is driven by the wrist temperature, which averages around 30° C. Based on the dose-responsive end-point signal of CoRPLA and a DNA hydrogel-based colorimetric patch, multi-level visual readout is realized within 25 minutes. The distinguishment of pathogen levels in at-home setting is necessary for the routine monitoring of some infectious diseases such as human papillomavirus (HPV), where the early- and late-stage of infection characterized by low- and high-viral loads require different medical treatments.

In certain embodiments, the subject methods pertain to an amplicon-free CRISPR-based one-pot loop-mediated isothermal amplification at low temperature range (30-37° C.) for point-of-care diagnosis of HPV-related cfDNA (16 E4) and other viral pathogens such as SARS-CoV-2 RNA, Influenza A (InfA), and human respiratory syncytial virus (RSV), Human papillomavirus (HPV), double-stranded DNA or other single-stranded DNA, including cancer-related cfDNA. In certain embodiments, amplicons of a target nucleic acid sequence, such as, for example, HPV-related cfDNA, are cleaved by cis cleavage activity of LbCas12a enzyme after its activation during the first 10-min of the target amplification.

In certain embodiments, the subject invention can deplete the amplicon-derived aerosol contamination for point-of-care diagnostics by destroying the valid primer binding sequence within the specific amplification product (amplicon) containing a “RPLA-like” loop structure using CRISPR/Cas enzyme cis cleavage activity that can be hybridized and amplified again with an RPA primer without the virus RNA target. In certain embodiments, the subject methods can reduce or eliminate false positive results.

In certain embodiments, the subject methods pertain to real-time colorimetric readout simultaneously with CoRPLA reaction with a glucose oxidase (GOx)-triggered semi-quantitative colorimetric pad by the in-situ release of the GOx reporter upon the trans-cleavage of the DNA hydrogel (DNAgel) scaffold during the CoRPLA reaction.

In certain embodiments, the subject methods pertain to an adjustable visual readout sensitivity by multiple test bands with different sensitivities by changing the amounts of the sodium 3,5-dichloro-2-hydroxybenzenesulfonate (DHBS) substrate in the colorimetric pad.

Advantageously, the subject invention is a programmable, low-cost, and easy-to-use platform offering a promising strategy for self-testing for disease monitoring and during viral pandemics is not limited by strict environmental control and professional operators. In addition, the subject invention can enhance the sensitivity of one-pot reactions by protecting and stimulating the initial stage of amplification by designing the sgRNA recognition site to cleave towards the fully extended amplicon repeats.

### BRIEF DESCRIPTION OF THE DRAWINGS

FIG. 1 illustrates the CRISPR-regulated amplicon-depleting mechanism by targeting the binding region of the “RPLA-like” bubble primer and the collateral cleavage activity towards the DNA scaffold in the DNA hydrogel (DNAgel) pad. Inspired by the explosive amplification model of RPLA, we designed a bubble primer for RPA which accelerates the amplification kinetics. With this design, we can fully exploit the cis- and trans-cleavage of the Cas12a enzyme. In the CoRPLA system, Cas12 cis cleavage is employed to cleave the amplicon into non-amplifiable small fragments once sufficient activator strands accumulate rapidly, thereby eliminating the risk of amplicon aerosol contamination.

FIG. 2 illustrates the workflow of self-testing using the CoRPLA-tape. Left panel: target pathogens for CoRPLA-tape testing in this work. Each pathogen was tested in one tape with semi-quantitative determination of viral load levels around 1000 cps/μL. Right upper: photographs of the integrated CoRPLA-tape biosensor on the wrist. Scale bar, 0.5 cm. T1 T2 are multi-level test bands by tuning the sensitivity.

FIGS. 3A-3E illustrate the design and performance of the bubble primer for RPLA and CoRPLA. FIG. 3A shows CoRPLA bubble primer design (upper) and reaction schematic (lower). Only bubble primers contain PAM sequences (yellow rectangle). Light gray circles: recombinase. Black circles: single-stranded DNA binding protein. The complementary sequence of the forward primer is highlighted in dark gray. Inverted repeat: the target sequence repeat on the same strand induced by the inverted loop structure. In our system, usually only one repeat is formed. FIG. 3B shows a schematic of the self-induced RPLA-like amplification at 37° C. Loop structures were intentionally introduced into the ssDNA template to mimic the intermediate dual loop structure in a conventional RPLA. Linear (left) and looped (right) primers were used in this system. Only reverse primer is illustrated for simplicity (labelled as P). FIG. 3C shows the product profile of the self-induced RPLA-like amplification with linear primer. Template amount is labeled at each lane. Each reaction contains 4 U Bst2.0, 1× NEBuffer 3.1, 1.4 mM dNTP, 1.35 μM of each primer. FIG. 3D shows the product profile of the self-induced RPLA-like amplification with looped primer. Template amount is labeled at each lane. Each reaction contains 4 U Bst2.0, 1× NEBuffer 3.1, 1.4 mM dNTP, 1.35 μM of each primer. FIG. 3E shows fragment analysis of the reaction product using bubble RPA primer compared to conventional linear RPA primer. FIG. 3F shows one-pot RPA real-time reaction signal using the bubble RPA primer with CRISPR/Cas12a system with PAM (left panel) and end-point fluorescence (right panel). FIG. 3G shows one-pot RPA real-time reaction signal using the linear RPA primer with CRISPR/Cas12a with PAM-less CRISPR system (left panel) and end-point fluorescence (right panel).

FIGS. 4A-4B illustrate the version 1 (v1) and version 2 (v2) sgRNA design (FIG. 4A) and the amplification product to be cleaved by Cas12 in these two designs (FIG. 4b). The sgRNA recognition sites are highlighted by the grey dashed rectangles. The loop structure in the bubble primers also impacts the binding of Cas12 by steric hindrance until it is opened by the primer extension by the reverse primers forming the inverted repeats. Thus, the PAM-containing bubble primer coupled with the v1 sgRNA design effectively keeps Cas12 inactive until primer extension.

FIG. 5 illustrates a shorter spacer length between the PAM sequence and the hairpin-loop structure within the bubble primer producing lower background signal. All the error bars indicate SEM, n=3. The hairpin-loop structure efficiently blocking the PAM and activator region from Cas12 by steric hindrance, specifically when the PAM region is located closer to the stem.

FIGS. 6A-6B illustrate fragment analysis of the reaction product using linear RPA primer design (FIG. 6A) and bubble RPA primer design (FIG. 6B). End-point fragment analysis further confirmed the complete depletion of the reaction products under these conditions.

FIGS. 7A-7B-FIG. 7A shows RPA real-time signal at 30° C. using 2 mM, 8 mM, 20 mM of Mg2+ (labelled as 2 Mg2+, 8 Mg2+, 20 Mg2+ in figure legend). 0.5 cps/μL of target DNA was used for the positive tests. FIG. 7B shows the product profile of 20-min RPA with different Mg2+ concentration analyzed by fragment analyzer. Both ssDNA and dsDNA fragments are visualized. Fragments in positions corresponding to dsDNA fragments with length between 200˜500 base-pairs are considered as amplification products. P: HPV16 E4 cfDNA present. N: HPV16 E4 cfDNA absent.

FIGS. 8A-8B-FIG. 8A shows fluorescence real-time reaction signal of CoRPLA assay at 25° C. or at 30° C. using the linear primer. FIG. 8B shows the fluorescence real-time reaction signal of CoRPLA assay at 25° C. or at 30° C. using the bubble primer. 0.5 cps/μL of target DNA was used for the positive tests.

FIGS. 9A-9B-FIG. 9A shows amplicon depletion performance of CoRPLA evaluated by fragment analysis. 10⁴ cps/μL of target was used in both tests. RPLA: RPA reaction without Cas12 using the bubble primer. FIG. 9B shows carryover contamination test for CoRPLA at 30° C. CoRPLA: carryover products from CoRPLA reaction added to the round 2 (R2) CoRPLA; NTC: non-target control of CoRPLA. RPA: carryover products from RPA reaction added to the R2 CoRPLA. 10⁴ cps/μL HPVE4 cfDNA template was used in round 1 reactions. Data shown are end-point signals after 20 minutes of R2 reactions.

FIGS. 10A-10B illustrate simulation of CoRPLA dose-responsive end point signal. FIG. 10A shows simulation of RPA dynamic based on the RPA dynamic model with adjustment in parameters to better describe kinetics in lower temperature (kb=10 molecules−1 s−1; kr=0.1 s−1; ka+=0.00001 molecules−1 s−1; ka−=0.0001 s−1). FIG. 10B shows prediction of CoRPLA signal generation. The kinetics of early trans-cleavage initiation and nucleic acid amplification suppression are not considered in this model.

FIGS. 11A-11B-FIG. 11A shows fluorescence-based CoRPLA reaction kinetics with different concentrations of HPV16 E4 cfDNA. FIG. 11B shows 20-min end-point fluorescence signal of CoRPLA tests of different concentration of HPV16 E4 cfDNA. Lg (conc) is the log 10 value of the target concentrations with units of cps/μL. The system successfully differentiated input target levels ranging from 0.5 cps/μL to 500 cps/μL. T-test between fluorescent signals of each test and that of NTC was conducted. Error bars indicate SEM (n=2-3). ****, P<0.0001; ***, 0.0001<P<0.001; **, 0.001<P<0.01; ns, non-significant, P>0.05.

FIGS. 12A-12C-FIG. 12A shows detection of SARS-CoV-2 pseudo virus in negative clinical sample background using RT-CoRPLA (5% glycerol was added to facilitate combination of the RT enzyme and primer with template). FIG. 12B shows qPCR and RPA primer Ct value validated by SARS-CoV-2 pseudo virus. FIG. 12C shows the selection of RT enzyme. Script IV and AMV are two potential candidates that demonstrated effectiveness in complex enzymatic one-pot reaction systems at a lower temperature (<37° C.) than the typical RT temperature of 42° C. When combined with reverse transcriptase, the CoRPLA assay (RT-CoRPLA) also detected SARS-CoV-2 viral RNA at 30° C. at concentrations as low as 1 cp/μL, showing the feasibility of CoRPLA as a deployable method for detecting various pathogens.

FIGS. 13A-13B illustrate DNA hydrogel enabling on-skin tape with real-time colorimetric nucleic acid detection. FIG. 13A shows the working principle of CRISPR/Cas-responsive colorimetric DNA hydrogel. FIG. 13B shows nucleic acid based CRISPR/Cas activation on colorimetric hydrogel. A Y-scaffold is used for stable gelation; linker can be optimized for different cleavage kinetics. Real-time DNA detection responsive colorimetric readout schematic. The colorimetric pad in which the GOx is sealed in the DNA hydrogel is directly assembled on top of a substrate layer containing the colorimetric readout reagents and the CoRPLA reagents in CoRPLA-tape as the test bands. The DNA scaffold within the hydrogel was assembled from double-stranded ‘Y’-motifs, which constituted the vertices of the scaffold, and single-stranded ‘linker’-motifs, which constituted the edges.

FIGS. 14A-14C-FIG. 14A shows the working principle of hydroxyl radicals activated chromogenic dye 3. FIG. 14B shows colorimetric readout sensitivity enhancement by adding phenol color-assist group 3,5-dichloro-2-hydroxy-benzenesulphonic acid (DHBS) to get quinoid product. 100% refers to 10 g GOx when 100% CRISPR/Cas enzyme cleavage efficiency. Right: 4-amino antipyrine (4-AAP) without DHBS. Left: sensitivity enhancement by adding DHBS. Sensitivity enhancement by adding a phenol color-assist group to produce quinoid product. The optimized testing buffer is 8 mM 4-AAP, 10 mg/mL HRP, 10 mM glucose, 0.3 mg/mL GOx. FIG. 14C. LFA-based CoRPLA fluorescence and colorimetric visualization.

FIGS. 15A-15E illustrate semi-quantitative response of CoRPLA enabled visual multi-level testing on lateral-flow testing strip. FIG. 15A shows Manufactured prototype of CoRPLA on a multiple-band lateral flow testing strip. FIG. 15B shows Visual results of semi-quantitative response on a test testing strip to different amount of GOx as a colorimetric initiator. T1: high-sensitivity band referring to level 1, T2: low-sensitivity band referring to level 2. FIG. 15C shows Gray scale of the of high/low sensitive bands induced by different GOx levels. FIG. 15D shows and level two (high GOx) (D) classification based on the colorimetric readout. The underlined grey value (80%) is the threshold for naked eye identification of the color change. The inset in FIG. 15E shows showed the 2-level test differentiating two different concentration ranges of viral targets (0.5-1000 and above 1000 cps/μL) and negative control. The numbers below the photographs of the test testing strips indicate the target amount used in the respective tests, with a unit of cps/μL. Error bars indicate SEM (n=3).

FIGS. 16A-16C illustrate pH value optimization of the DNA hydrogel. FIG. 16A shows DNA hydrogel motif Y-scaffold and L-linker annealing validation. The band position is consistent with the expected sizes of the DNA structures, and the intensity showed that the Y-shaped DNA scaffolds and L-shaped linkers were successfully formed and had good stability. FIG. 16B shows gelation under different pH conditions. At high pH, DNA hydrogel easily dissolved. At pH 7.5: The double-stranded structure of DNA usually unwinds with destroyed hydrogen bonds and base pair interactions. DNA molecules maintain a stable double-stranded structure. FIG. 16C shows GOx-based colorimetric leakage analysis work for DNA hydrogel. At lower pH, DNA hydrogel is formed, but leakage of red dye can be observed due to unstable hydrogel network.

FIGS. 17A-17D-FIG. 17A shows gelation characterization of the DNA scaffold and linkers. Formation of double stranded structure was visualized by fluorescence from SYBR-Green dsDNA dye. The fluorescence was emitted under LED 450 nm (upper) or confocal LD 500 nm (lower). Confocal microscopy was used to observe the porous structure of formed DNA scaffold. FIG. 17B shows hybrid additives selection to create double-network hydrogel with different pore size to optimize the leakage in inactivated reaction. The lower panel shows the SEM image of the DNA hydrogel with the corresponding additives. PVA: polyvinyl alcohol, PVP: polyvinylpyrrolidone. Scale bar=100 μm. FIG. 17C shows repeatable on-skin positive visual readout using mock sample inputs with 10 cps/μL synthetic DNA. Surface modification on nitrocellulose membrane at the conjugate pad with an Anti-6-FAM antibody tagged with FAM-labeled reporter. FIG. 17D shows CoRPLA on-skin test with DNA hydrogel-responsive colorimetric readout. The lower panel showed the enhancement in the colorimetric readout by the introduction of the hydrophobic barrier (HB zone) with methylsilsequioxane (MSQ).

FIG. 18 illustrates the CoRPLA-tape. Left: Schematic of CoRPLA-tape. I. Medical skin-adherent tape; II. Sealing; III. Sample pad; IV. Conjugation pad; V. DNA hydrogel pad; VI. Colorimetric pad; VII. Adsorption pad; VIII. Nitrocellulose (NC) membrane; IX. Adhesive backing. III and IV form the sampling zone. V, VI, and VIII form the DNA/RNA testing zone. Right: The mechanism of on-skin tape for DNA/RNA assay. It involves fully automatic sample routing, reagent activation, and real-time readout. Gray arrows represent the flow direction of the DNA/RNA target and lysed saliva liquid mixture. The glowing blue color indicates the activated enzyme in the reaction zone. The purple color indicates the color-changing test bands initiated by the vertically released GOx from the DNAgel-coated reaction zone.

FIGS. 19A-19B-FIG. 19A shows clinical validation of CoRPLA-tape. Lower test bands: high-sensitivity band referring to level 1. Higher test bands: low-sensitivity band referring to level 2. Cases labelled with grey color without a Ct value are negative cases in the corresponding testing. CoV: SARS-CoV-2; InfA: influenza A; RSV: human respiratory syncytial virus; HPV16: human papillomavirus type 16. FIG. 19B shows photograph of integrated CoRPLA-tape on wrist. Lower panel shows an example of wrist temperature. During the clinical validation, we tuned the sensitivities of the two test bands in the two-level CoRPLA tape to distinguish the high viral load (Ct<30) and the low viral load (40>Ct>30) positive samples. The two-level testing results of clinical samples using CoRPLA-tape were consistent with the Ct values of the samples, both in RNA and DNA testing.

FIGS. 20A-20C illustrate deep learning assisted machine learning-based software for result interpretation of the semi-quantitative self-test. FIG. 20A shows the workflow of the proposed method. FIG. 20B shows the principle of the segmentation model to produce the segmented band. Machine learning assisted 2D clustering for diagnostic multi-level output using agglomerative clustering classification model. Left panel: example of test bands filtered by the model. Center panel: Validation of the model using detection results during CoRPLA optimization. Right panel: Result interpretation of CoRPLA-tape testing 31 clinical samples. FIG. 20C shows control band, T1: high-sensitivity band referring to level 1, T2: low-sensitivity band referring to level 2. Each point in the plot indicates one test. Yellow: negative. Purple: low viral load positive. Cyan: high viral load positive.

FIG. 21 illustrates deep learning-based image filtration for downstream disease level classification. The initial photos were first segmented into the same size with single color band using YOLOv5, a widely used deep-learning model which facilitated precise extraction of image features 48 i.e., the test bands on the CoRPLA testing strip.

FIGS. 22A-22C illustrate model training metrics. FIG. 22A shows convergence analysis. FIG. 22B shows confusion analysis of the true data and the predicted data with 98% accuracy in 1000 trials on the true positive trials. FIG. 22C shows confidence and recall analysis of the classification model.

FIGS. 23A-23B-FIG. 23A shows the principle of antigen recognition by antibody-oligo activator on NC membrane testing testing strip. FIG. 23B shows the real-time fluorescence signal from CoRPA on-chip elisa assay with 1 nM antigen as positive control.

FIG. 24 illustrates numerical simulation method of loop structure.

FIGS. 25A-25B illustrates skin-mimic experiments for interstitial field sampling. FIG. 25A shows 4% agarose gel-based skin-mimic extraction efficiency by qPCR. FIG. 25B shows 30° C. CoRPA signal from skin-mimic gel-based extracted HPV E4 cfDNA (Inset: repeatable visual signal). Glucose is successfully detected under mock skin to mimic two bands (T1 and T2) semi quantitative visual readout after CRISPR/Cas activation for GOx labelled ssDNAreporter cleavage.

FIGS. 26A-26B FIG. 26A shows the 4% agarose gel-based skin-mimic extraction efficiency by qPCR. FIG. 26B shows the 30-degree coRPA signal from skin-mimic gel-based extracted HPV E4 cfDNA (Inset: repeatable visual signal).

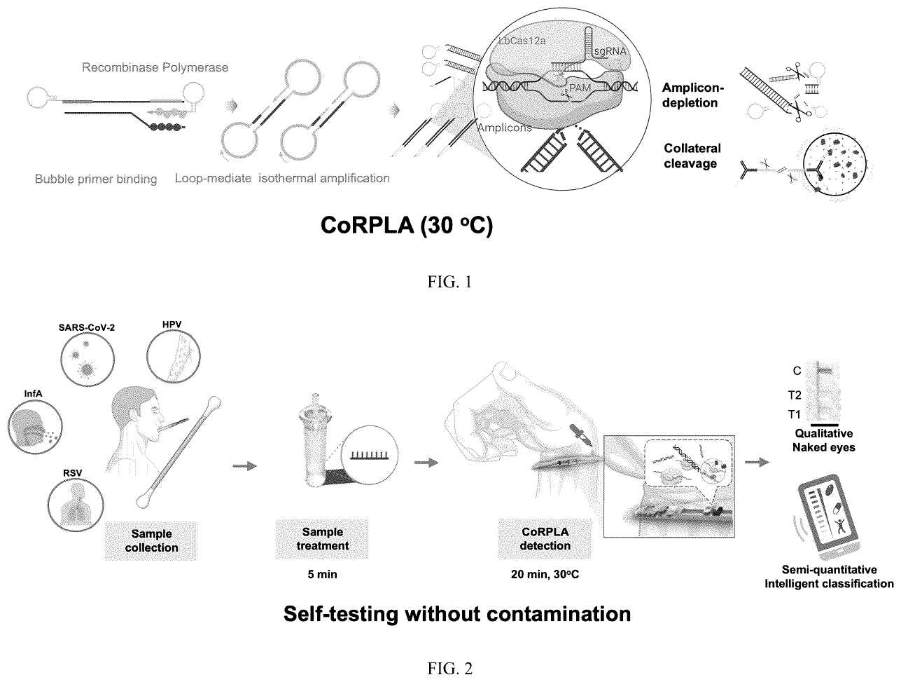

### BRIEF DESCRIPTION OF THE SEQUENCES

SEQ ID NO: 1: HPV16 E4 CoRPLA crRNA PAM-related-1
SEQ ID NO: 2: HPV16 E4 FP
SEQ ID NO: 3: HPV16 E4 BP
SEQ ID NO: 4: HPV16 E4 Bubble FP-510-PAM in stem
SEQ ID NO: 5: HPV16 E4 Bubble BP-510-PAM in stem
SEQ ID NO: 6: HPV16 E4 Bubble FP-720-PAM in stem
SEQ ID NO: 7: HPV16 E4 Bubble BP-720-PAM in stem
SEQ ID NO: 8: HPV16 E4 Bubble FP-720-PAM in primer
SEQ ID NO: 9: HPV16 E4 Bubble BP-720-PAM in primer
SEQ ID NO: 10: HPV16 E4 cfDNA sequence
SEQ ID NO: 11: SARS-CoV-2 N gene crRNA-PAM-free
SEQ ID NO: 12: SARS-CoV-2 N gene FP
SEQ ID NO: 13: SARS-CoV-2 N gene BP
SEQ ID NO: 14: SARS-CoV-2 N gene Bubble FP
SEQ ID NO: 15: SARS-CoV-2 N gene Bubble BP
SEQ ID NO: 16: SARS-CoV-2 N Gene Pseudovirus
SEQ ID NO: 17: InfA M gene FP
SEQ ID NO: 18: InfA M gene BP
SEQ ID NO: 19: InfA M gene Bubble FP
SEQ ID NO: 20: InfA M gene Bubble BP
SEQ ID NO: 21: InfA M gene-crRNA
SEQ ID NO: 22: RSV N gene FP
SEQ ID NO: 23: RSV N gene BP
SEQ ID NO: 24: RSV N gene Bubble FP
SEQ ID NO: 25: RSV N gene Bubble BP
SEQ ID NO: 26: RSV N gene-crRNA
SEQ ID NO: 27: ssFQ-HEX
SEQ ID NO: 28: ssFQ-FAM

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. The term nucleic acid is used interchangeably with gene, cDNA, and mRNA encoded by a gene.

As used herein, the term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

In this application, the terms “polypeptide”, “peptide”, and “protein” are used interchangeably herein to refer to a polymer of amino acids. The terms apply to amino acid polymers in which one or more amino acid residues are artificial chemical mimetic of a corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

The term “organism” as used herein includes viruses, bacteria, fungi, plants and animals. Additional examples of organisms are known to a person of ordinary skill in the art and such embodiments are within the purview of the materials and methods disclosed herein. The assays described herein can be useful in analyzing any genetic material obtained from any organism.

The term “genome”, “genomic”, “genetic material” or other grammatical variation thereof as used herein refers to genetic material from any organism. A genetic material can be viral genomic DNA or RNA, nuclear genetic material, such as genomic DNA, or genetic material present in cell organelles, such as mitochondrial DNA or chloroplast DNA. It can also represent the genetic material coming from a natural or artificial mixture or a mixture of genetic material from several organisms.

As used herein, “a target region” or “a target sequence” is a region of interest in genetic material of an organism.

The term “hybridizes with” when used with respect to two sequences indicates that the two sequences are sufficiently complementary to each other to allow nucleotide base pairing between the two sequences. Sequences that hybridize with teach other can be perfectly complementary but can also have mismatches to a certain extent. Therefore, the sequences at 5′ and 3′ ends of the extension and ligation probes described herein may have a few mismatches with the corresponding target sequences at 5′ and 3′ ends of the target genomic region as long as the extension and the ligation probes can hybridize with the target sequences to facilitate capturing of the target genomic region. Depending upon the stringency of hybridization, a mismatch of up to about 5% to 20% between the two complementary sequences would allow for hybridization between the two sequences. Typically, high stringency conditions have higher temperature and lower salt concentration and low stringency conditions have lower temperature and higher salt concentration. High stringency conditions for hybridization are preferred, and therefore, the sequences at 3′ and 5′ ends of the extension and ligation probes are preferred to be perfectly complementary to the corresponding target sequences at 3′ and 5′ ends of the target genomic region.

Throughout this disclosure, different sequences are described by specific nomenclature, for example, a RPLA primer sequence, PAM code sequence, and target sequence. When such nomenclature is used, it is understood that the identified sequence is substantially identical or substantially reverse complementary to at least a part of the corresponding sequence. For example, “a RPLA primer sequence” describes a sequence that is substantially identical to at least a part of the RPLA primer sequence or substantially reverse complementary to at least a part of the primer RPLA sequence. This is because when a captured target genomic region is converted into a double stranded form comprising the primer binding sequence, the double stranded target genomic region can be sequenced using a primer having a sequence that substantially identical or substantially reverse complementary to at least a part of primer binding sequence. Thus, the nomenclature is used herein to simplify the description of different polynucleotides and parts of polynucleotides used in the methods disclosed here; however, a person of ordinary skill in the art would recognize that appropriate substantially identical or substantially reverse complementary sequences to at least a part of the corresponding sequences could be used to practice the methods disclosed herein.

Also, two sequences that correspond to each other, for example, a target sequence and a primer sequence, have at least 90% sequence identity, preferably, at least 95% sequence identity, even more preferably, at least 97% sequence identify, and most preferably, at least 99% sequence identity, over at least 70%, preferably, at least 80%, even more preferably, at least 90%, and most preferably, at least 95% of the sequences. Alternatively, two sequences that correspond to each other are reverse complementary to each other and have at least 90% perfect matches, preferably, at least 95% perfect matches, even more preferably, at least 97% perfect matches, and most preferably, at least 99% perfect matches in the reverse complementary sequences, over at least 70%, preferably, at least 80%, even more preferably, at least 90%, and most preferably, at least 95% of the sequences. Thus, two sequences that correspond to each other can hybridize with each other or hybridize with a common reference sequence over at least 70%, preferably, at least 80%, even more preferably, at least 90%, and most preferably, at least 95% of the sequences. Preferably, two sequences that correspond to each other are 100% identical over the entire length of the two sequences or 100% reverse complementary over the entire length of the two sequences.

As used herein “CRISPR” (Clustered Regularly Interspaced Short Palindromic Repeat”) refers to a family of DNA sequences found in the genomes of prokaryotic organisms that provide adaptive immunity against foreign elements. They are used to detect and destroy DNA in an organism's cells.

As used herein “CRISPR/Cas9” is a gene editing technology that utilize non-homologous end joining and homology-directed repair for DNA repair mechanism.

As used herein “one pot detection” refers to a method of combining substances where it is all in a single closed tube together.

As used herein “loop-mediated isothermal amplification” (RPLA) is a single-tube technique for the amplification of DNA and a tool to detect certain diseases. RPLA uses about 2 primers recognizing about 6 to about 8 distinct regions of target DNA to make a specific amplification reaction.

As used herein “amplicon” is a piece of DNA or RNA that is the source and/or product of amplification and/or replication events.

All references cited herein are hereby incorporated by reference in their entirety.

The subject invention relates to a skin-temperature triggered amplicon-depleted scheme that utilizes CRISPR/Cas enzyme specific sequence recognition towards the primer binding region in the CRISPR based one-pot recombinase polymerase loop-mediated amplification named CoRPLA to accomplish a promising strategy for low-cost, equipment-free, multi-level self-testing for disease monitoring and during viral pandemics in non-lab environments.

The subject invention is designed to solve the contamination issue for point-of care diagnostics by destroying the amplicons primer binding sequence after the amplification initiation stage that can be hybridized and amplified again by the primer without virus RNA target, which is crucial to combat false positive result in negative samples. Advantageously, the subject invention is environmentally friendly and user-friendly. In addition, the subject invention can solve the sensitivity issue in other one-pot reaction studies resulting in the loss of the target template to some extend at the initial stage of amplification due to the cis cleavage towards the target sequence even before the target amplification region.

In certain embodiments, the subject invention provides compounds for Amplicon-depleted CRISPR/Cas-regulated isothermal molecular assay at skin temperature enabling multi-level, equipment-free, home-based self-testing of viral infections.

In certain embodiments, the subject invention provides novel oligonucleotides for amplicon-free CRISPR-based one-pot detection of nucleic acid sequences with RPLA. The subject invention provides a series of at least 4, 5, 6, 7, 8, 9, 10 or more RPLA primers that can be used in the subject methods. In certain embodiments, the at least 4 primers comprise a forward outer primer, forward primers, backward outer primer, and backward primers. In certain embodiments, each primer can hybridize to target sequence, in which the target sequence is derived from genetic material from an organism or virus of interest, such as, for example, SARS-CoV-2.

In certain embodiments, the RPLA primer can encode a site for small guide RNA (sgRNA) recognition, preferably in a primer binding region that results from the hybridization and extension of a forward or backward primers. In certain embodiments, the RPLA primers of the subject invention can comprise a specific sequence for LbCas12a sgRNA recognition (sgRNA features are highlighted in Table SEQ ID NO: 1, 11, 21, 26) as both comprise an activation step and an amplified product cleaning step. In certain embodiments, the RPLA forward or backward primers can be designed with a PAM code (TTC in Table 1 SEQ ID NO: 4-9) about 40 bases (20-50 bases range is recommended) downstream of the sequence that is complementary to the target sequence. In certain embodiments, the PAM code sequence is immediately downstream of the complementary sequence or at least 1, 2, 3, 4, 5, 6, 7, 8, 9, 10 or more nucleotides downstream of the complementary sequence. In certain embodiments, the amplification reaction can be transduced to a CRISPR signal amplification. In certain embodiments, CRISPR/Cas can be activated by specific cis cleavage activity on the target DNA and then CRISPR/Cas can cleave the surrounding single stranded DNA that serve as reporters.

In certain embodiments, the LbCas12a enzyme sgRNA recognizes and hybridizes to the pair chain containing the complementary sequence of primer binding regions after the target amplification, which will become the extended double stranded DNA repeat after the loop region of the RPLA dumbbell structure. In certain embodiments, the Cas12a enzyme can use the sgRNA recognition site on the primer sequence that is exponentially amplified after the target binding initiation stage. In certain embodiments, the recognition site of the sgRNA can be different than the primer binding site. In certain embodiments, the recognition site of the sgRNA can be a sequence complementary with the target nucleotide sequence and about 20 bases downstream of the forward or backward primer binding region, which is within the region in the dumbbell structure produced by the extension of the primer. In certain embodiments, a PAM code is sited about 40 bases downstream (20-50 bases range is recommended) of the target for CRISPR/Cas recognition.

In certain embodiments, the CRISPR recognition sequence can be designed in recombinase polymerase amplification using a PAM-free seed region which is within about 3 to 10 bases downstream to downstream to a protospacer adjacent motif (PAM) by modifying a TTN PAM code in the stem region (7 bp) of a bubble primer for CRISPR/Cas recognition.

In certain embodiments, the amplification of the target sequence can use probes to identify and/or quantify the amplified target sequence. In certain embodiments, the probes can produce a signal generated from a single-stranded and/or double-stranded DNA reporter, such as, for example, a single-stranded DNA fluorescence reporter (ssFQ). In certain embodiments, probes can be used to produce a signal generated from a double or single-stranded DNA methylene blue reporter (dsMB/ssMB). In certain embodiments, the fluorophore is at 3′ end and quencher is at the 5′ end respectively with a 5 unit single stranded DNA sequence, such as, for example, TTTTT, which is optimized to achieve highest signal in the middle. In other embodiments, the fluorescence signal is generated from excitation of cleaved fluorophore labelled reporters. Further, the simple visual readout is realized by an optical filter on the top of the dark chamber to filtrate the extra lights with a wavelength other than that of the emission light (520 nm to 530 nm) to distinguish positive signal (green color) and negative (dark background color).

In certain embodiments, fluorophores are used as labels to generate a fluorescently labeled probe included in embodiments of methods and compositions of the present invention can be any of numerous fluorophores including, but not limited to, 4-acetamido-4′-isothiocyanatostilbene-2,2′ disulfonic acid; acridine and derivatives such as acridine and acridine isothiocyanate; 4-amino-N-[3-vinylsulfonyl)phenyl]naphthalimide-3,5 disulfonate, Lucifer Yellow VS; N-(4-anilino-1-naphthyl) maleimide; anthranilamide, Brilliant Yellow; BIODIPY fluorophores (4,4-difluoro-4-bora-3a,4a-diaza-s-indacenes); coumarin and derivatives such as coumarin, 7-amino-4-methylcoumarin (AMC, Coumarin 120), 7-amino-4-trifluoromethylcoumarin (Coumarin 151); cyanosine; DAPDXYL sulfonyl chloride; 4′,6-diaminidino-2-phenylindole (DAPI); 5′,5″-dibromopyrogallol-sulfonphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-4′-dimethylaminophenylazo)benzoic acid (DABCYL); 4-dimethylaminophenylazophenyl-4′-isothiocyanate (DABITC); EDANS (5-[(2-aminoethyl)amino]naphthalene-1-sulfonic acid), eosin and derivatives such as eosin isothiocyanate; erythrosin and derivatives such as erythrosin B and erythrosin isothiocyanate; ethidium such as ethidium bromide; fluorescein and derivatives such as 5-carboxyfluorescein (FAM), hexachlorofluorescein (HEX), 5-(4,6-dichlorotriazin-2-yl)aminofluorescein (DTAF), 2′,7′-dimethoxy-4′,5′-dichloro-6-carboxyfluorescein (JOE) and fluorescein isothiocyanate (FITC); fluorescamine; green fluorescent protein and derivatives such as EBFP, EBFP2, ECFP, and YFP; IAEDANS (5-({2-[(iodoacetyl)amino]ethyl}amino) naphthalene-1-sulfonic acid), Malachite Green isothiocyanate; 4-methylumbelliferone; orthocresolphthalein; nitrotyrosine; pararosaniline; Phenol Red; B-phycoerytnin; 0-phthaldialdehyde; pyrene and derivatives such as pyrene butyrate, 1-pyrenesulfonyl chloride and succinimidyl 1-pyrene butyrate; QSY 7; QSY 9; Reactive Red 4 (Cibacron® Brilliant Red 3B-A); rhodamine and derivatives such as 6-carboxy-X-rhodamine (ROX), 6-carboxyrhodamine (Rhodamine 6G), rhodamine isothiocyanate, lissamine rhodamine B sulfonyl chloride, rhodamine B, rhodamine 123, sulforhodamine B, sulforhodamine 101 and sulfonyl chloride derivative of sulforhodamine 101 (Texas Red); N,N,N′,N-tetramethyl-carboxyrhodamine (TAMRA); tetramethyl rhodamine; tetramethyl rhodamine isothiocyanate (TRITC); riboflavin; rosolic acid; and terbium chelate derivatives.

In certain embodiments, the fluorescent moiety can comprise a fluorescent protein, such as, for example, a green fluorescent protein (GFP); a modified derivative of GFP, including, for example, a GFP comprising S65T, an enhanced GFP; blue fluorescent protein, including, for example, EBFP, EBFP2, Azurite, and mKalamal; cyan fluorescent protein, such as, for example, ECFP, Cerulean, CyPet, mTurquoise2; and yellow fluorescent protein derivatives such as, for example, YFP, Citrine, Venus, YPet.

Exemplary quencher labels include a fluorophore, a quantum dot, a metal nanoparticle, and other related labels. Suitable quenchers include Black Hole Quencher®-1 (Biosearch Technologies, Novato, CA), BHQ-2, Dabcyl, Iowa Black® FQ (Integrated DNA Technologies, Coralville, IA), IowaBlack RQ, QXL™ (AnaSpec, Fremont, CA), QSY 7, QSY 9, QSY 21, QSY 35, IRDye QC, BBQ-650, Atto 540Q, Atto 575Q, Atto 575Q, MGB 3′ CDPI3, and MGB-5′ CDPI3. In one instance, the term “quencher” refers to a substance which reduces emission from a fluorescent donor when in proximity to the donor. In preferred embodiments, the quencher is within 10, 11, 12, 13, 14, 15, 16, 17, 18, 19, 20, 21, 22, 23, 24, 25, 26, 27, 28, 29 or 30 nucleotide bases of the fluorescent label. Fluorescence is quenched when the fluorescence emitted from the fluorophore is detectably reduced, such as reduced by 10%, 20%, 30%, 40%, 50%, 60%, 70%, 80%, 90%, 95%, 99% or more.

In certain embodiments, the concentration of the fluorescent probe in the compositions and method of use is about 0.01 μM to about 1000 μM, about 0.1 μM to about 100 μM, about 0.1 μM to about 50 μM, about 0.1 μM to about 10 μM, or about 0.5 μM to about 3 μM. In certain embodiments, the concentration of the fluorescent probe is about 0.01 μM, about 0.1 μM, 0.2 μM, about 0.25μ, about 0.3μ, about 0.4μ, about 0.5μ, about 0.6μ, about 0.7μ, about 0.8μ, about 0.9μ, about 1μ, about 1.5μ, about 2μ, about 2.5μ, or about 3 μM.

In certain embodiments, dyes ranging between 500 nm to 700 nm have the advantage of being in the visible spectrum and can be detected using existing photomultiplier tubes. In some embodiments, the broad range of available dyes allows selection of dye sets that have emission wavelengths that are spread across the detection range. Detection systems capable of distinguishing many dyes are known in the art. In some embodiments, the visual fluorescence signal can be seen by the naked eye or taken a picture of by smartphone.

In certain embodiments, the composition of the subject invention can contain other compounds, such as fluorophores, oligonucleotides, preservatives, buffers, and any combination thereof. These compounds can be added to the composition can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total composition.

The subject invention also relates to detection testing strips. The testing strip includes freeze dried oligonucleotide probes and primers, packaged into suitable packaging material, optionally in combination with instructions for using the testing strip components, e.g., instructions for performing a method of the invention. In one embodiment, a testing strip includes an amount of an oligonucleotide's probes and primers, and instructions for running the assay on a label or packaging insert. In further embodiments, a testing strip includes an article of manufacture, for performing the assay. Preferably, said testing strip comprises at one primer pair according to the invention. Said testing strip comprises more than one probe, e.g., at least two, at least three, at least four, at least five, at least six, at least 7, at least 8, at least 9, or at least 10 different probes, notably when the testing strip is intended to discriminate between different SARS-CoV-2 types or other infectious agents or genetic variations that cause disease.

In the testing strip according to the invention, the oligonucleotides (primers, probes) can be either kept separately, or partially mixed, or totally mixed. In a preferred embodiment, the testing strip according to the invention can also contain further reagents suitable for a PCR or RT-PCR step.

Such reagents are known to those skilled in the art, and include water, like nuclease-free water, RNase free water, DNAse-free water, PCR-grade water; salts, like magnesium, magnesium chloride, potassium; buffers such as Tris; enzymes, including polymerases, such as Taq, Vent™, Pfu, activatable polymerase, reverse transcriptase, and the like; nucleotides like deoxynucleotides, dideoxynucleotides, dNTPs, dATP, dTTP, dCTP, dGTP, dUTP; other reagents, like DTT and/or RNase inhibitors; and polynucleotides like polyT, polydT, and other oligonucleotides, e.g., primers.

In another preferred embodiment, the testing strip according to the invention comprises PCR controls. Such controls are known in the art, and include qualitative controls, positive controls, negative controls, internal controls, quantitative controls, internal quantitative controls, as well as calibration ranges. The internal control for said PCR step can be a template which is unrelated to the target template in the PCR step. Such controls also may comprise control primers and/or control probes. For example, in the case of SARS-CoV-2 detection, it is possible to use as an internal control, a polynucleotide chosen within a gene whose presence is excluded in a sample originating from a human body (for example, from a plant gene), and whose size and GC content is equivalent to those from the target sequence. In other embodiments, a positive control is included which comprises a polynucleotide sequence associated with the target nucleotide sequence, such as an unmutated portion of the target nucleotide sequence (or amplicon). In some embodiments, the positive control is amplified by the oligonucleotide primer pair used to amplify the target nucleic acid sequence. By way of example, positive control sequences for SARS-CoV-2 can be portions of the envelope, membrane, nucleocapsid gene, or invariant regions of the gene encoding the spike protein. For cells derived from specific human tissues, the positive control could be, for example, a portion of the following: the beta-actin gene, the aldolase gene, the dihydrofolate reductase gene, the glyceraldehyde phosphate dehydrogenase gene, the histone 3.3 gene, the hypoxanthine phosphoribosyltransferase gene, the Abelson gene (ABL), the BCR gene, the porphobilinogen deaminase gene (PBGD), or the beta-2-microglobulin gene (β2-MG).

In a preferred embodiment, the testing strip according to the invention contains means for extracting and/or purifying nucleic acid from a biological sample, e.g., from blood, serum, plasma, saliva, or nasal secretions. Such means are well known to those skilled in the art.

In a preferred embodiment, the testing strip according to the invention contains instructions for the use thereof. Said instructions can advantageously be a leaflet, a card, or the like. Said instructions can also be present under two forms: a detailed one, gathering exhaustive information about the testing strip and the use thereof, possibly also including literature data; and a quick-guide form or a memo, e.g., in the shape of a card, gathering the essential information needed for the use thereof. Instructions can therefore include instructions for practicing any of the methods of the invention described herein. For example, compositions can be included in a container, pack, or dispenser together with instructions for performing the nucleotide detection assay. Instructions may additionally include storage information, expiration date, or any information required by regulatory agencies such as the Food and Drug Administration or European Medicines Agency for use with a human or animal subject. The instructions may be on “printed matter,” e.g., on paper or cardboard within the testing strip, on a label affixed to the testing strip or packaging material or attached to a vial or tube containing a component of the testing strip. Instructions may comprise voice or video tape and additionally be included on a computer readable medium, such as a disk (floppy diskette or hard disk), optical CD such as CD- or DVD-ROM/RAM, magnetic tape, flash storage, electrical storage media such as RAM and ROM and hybrids of these such as magnetic/optical storage media.

In a preferred embodiment, said self-testing tape is a diagnostic testing strip, especially an in vitro diagnostic testing strip, i.e., a SARS-CoV-2 diagnostics testing strip.

TABLE 1
Sequence Table

| No. | Label | Sequence (5′ to 3′) | Units |
|---|---|---|---|
| SEQ ID NO: 1 | HPV16E4 | UAAUUUCUACUAAGUGUAGAUAGAGGAUAC | 41 |
| | CORPLA | UUCGUCGCUGC | |
| | crRNA PAM- | | |
| | related-1 | | |
| SEQ ID NO: 2 | HPV16E4 FP | AAGTATCCTCTGCTGAAATTATTAGGC | 27 |
| SEQ ID NO: 3 | HPV16E4 BP | CTATTACAGTTAATCCGTGCTTTGTGTG | 28 |
| SEQ ID NO: 4 | HPV16E4 Bubble FP-510- | 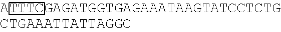 | 47 |
| | PAM in stem | | |
| SEQ ID NO: 5 | HPV16E4 Bubble BP-510- | 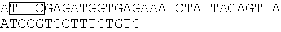 | 48 |
| | PAM in stem | | |
| SEQ ID NO: 6 | HPV16E4 Bubble FP-720- PAM in stem | 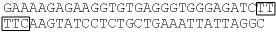 | 61 |
| SEQ ID NO: 7 | HPV16E4 Bubble BP-720- PAM in stem | 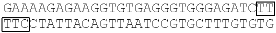 | 62 |
| SEQ ID NO: 8 | HPV16E4 Bubble FP-720- PAM in primer | 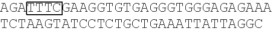 | 61 |
| SEQ ID NO: 9 | HPV16E4 Bubble BP-720- PAM in primer | 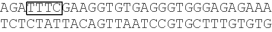 | 62 |
| SEQ ID NO: 10 | HPV 16 E4 | TATTATGTCCTACATCTGTGTTTAGCAGCGAC | 288 |
| | cfDNA | GAAGTATCCTCTGCTGAAATTATTAGGCAGCA | |
| | sequence | CTTGGCCAACCACTCCGCCGCGACCCATCCCA | |
| | | AAGCCGTCGCCTTGGGCACCAAAGAATCACA | |
| | | GACGACTATCCAGCGACCAAGATCAGAGCCA | |
| | | GACACCGGAAACCCCTGCCACACCAATAAGT | |
| | | TGTTGCACAGAGACTCAGTGGACAGTGCTCCA | |
| | | ATTCTCACTGCAGTTAACAGCTCACACAAAGC | |
| | | ACGGATTAACTGTAATAGTAACACTACACCCA | |
| | | TAG | |
| SEQ ID NO: 11 | SARS-CoV-2 N | UAAUUUCUACUAAGUGUAGAUAUUUUACCG | 41 |
| | gene crRNA- | UCACCACCACG | |
| | PAM-free | | |
| SEQ ID NO: 12 | SARS-CoV-2 N | CCAAGGTTTACCCAATAATACTGCGTCT | 28 |
| | gene FP | | |
| SEQ ID NO: 13 | SARS-CoV-2 N | TCTTTCATTTTACCGTCACCACCACGA | 27 |
| | gene BP | | |
| SEQ ID NO: 14 | SARS-CoV-2 N | AGATTTCGAAGGTGTGAGGGTGGGAGAGAAA | 62 |
| | gene Bubble FP | TCTCCAAGGTTTACCCAATAATACTGCGTCT | |
| SEQ ID NO: 15 | SARS-CoV-2 N | AGATTTCGAAGGTGTGAGGGTGGGAGAGAAA | 62 |
| | gene Bubble BP | TCTATCTTGGACTGAGATCTTTCATTTTACC | |
| SEQ ID NO: 16 | SARS-CoV-2 N | AUGUCUGAUAAUGGACCCCAAAAUCAGCGA | |
| | Gene | AAUGCACCCCGCAUUACGUUUGGUGGACCCU | |
| | Pseudovirus | CAGAUUCAACUGGCAGUAACCAGAAUGGAG | |
| | | AACGCAGUGGGGCGCGAUCAAAACAACGUC | |
| | | GGCCCCAAGGUUUACCCAAUAAUACUGCGUC | |
| | | UUGGUUCACCGCUCUCACUCAACAUGGCAAG | |
| | | GAAGACCUUAAAUUCCCUCGAGGACAAGGC | |
| | | GUUCCAAUUAACACCAAUAGCAGUCCAGAU | |
| | | GACCAAAUUGGCUACUACCGAAGAGCUACCA | |
| | | GACGAAUUCGUGGUGGUGACGGUAAAAUGA | |
| | | AAGAUCUCAGUCCAAGAUGGUAUUUCUACU | |
| | | ACCUAGGAACUGGGCCAGAAGCUGGACUUCC | |
| | | CUAUGGUGCUAACAAAGACGGCAUCAUAUG | |
| | | GGUUGCAACUGAUGGGAGCCUUGAAUACAC | |
| | | CAAAAGAUCACAUUGGCACCCGCAAUCCUGC | |
| | | UAACAAUGCUGCAAUCGUGCUACAACUUCCU | |
| | | CAAGGAACAACAUUGCCAAAAGGCUUCUAC | |
| | | GCAGAAGGGAGCAGAGGCGGCAGUCAAGCC | |
| | | UCUUCUCGUUCCUCAUCACGUAGUCGCAACA | |
| | | GUUCAAGAAAUUCAACUCCAGGCAGCAGUA | |
| | | GGGGAACUUCUCCUGCUAGAAUGGCUGGCA | |
| | | AUGGCGGUGAUGCUGCUCUUGCUUUGCUGC | |
| | | UGCUUGACAGAUUGAACCAGCUUGAGAGCA | |
| | | AAAUGUCUGGUAAAGGCCAACAACAACAAG | |
| | | GCCAAACUGUCACUAAGAAAUCUGCUGCUG | |
| | | AGGCUUCUAAGAAGCCUCGGCAAAAACGUA | |
| | | CUGCCACUAAAGCAUACAAUGUAACACAAGC | |
| | | UUUCGGCAGACGUGGUCCAGAACAAACCCAA | |
| | | GGAAAUUUUGGGGACCAGGAACUAAUCAGA | |
| | | CAAGGAACUGAUUACAAACAUUGGCCGCAA | |
| | | AUUGCACAAUUUGCCCCCAGCGCUUCAGCGU | |
| | | UCUUCGGAAUGUCGCGCAUUGGCAUGGAAG | |
| | | UCACACCUUCGGGAACGUGGUUGACCUACAC | |
| | | AGGUGCCAUCAAAUUGGAUGACAAAGAUCC | |
| | | AAAUUUCAAAGAUCAAGUCAUUUUGCUGAA | |
| | | UAAGCAUAUUGACGCAUACAAAACAUUCCC | |
| | | ACCAACAGAGCCUAAAAAGGACAAAAAGAA | |
| | | GAAGGCUGAUGAAACUCAAGCCUUACCGCA | |
| | | GAGACAGAAGAAACAGCAAACUGUGACUCU | |
| | | UCUUCCUGCUGCAGAUUUGGAUGAUUUCUC | |
| | | CAAACAAUUGCAACAAUCCAUGAGCAGUGC | |
| | | UGACUCAACUCAGGCCUAA | |
| SEQ ID NO: 17 | InfA M gene FP | ACCGAGGTCGAAACGTACGTTCTTTCTATC | 30 |
| SEQ ID NO: 18 | InfA M gene BP | CTACGCTGCAGTCCTCGCTCACTGGGCACG | 30 |
| SEQ ID NO: 19 | InfA M gene | AGATTTCGAAGGTGTGAGGGTGGGAGAGAAA | 64 |
| | Bubble FP | TCTACCGAGGTCGAAACGTACGTTCTTTCTAT | |
| | | C | |
| SEQ ID NO: 20 | InfA M gene | AGATTTCGAAGGTGTGAGGGTGGGAGAGAAA | 64 |
| | Bubble BP | TCTCTACGCTGCAGTCCTCGCTCACTGGGCAC | |
| | | G | |
| SEQ ID NO: 21 | InfA M gene- | UAAUUUCUACUAAGUGUAGAUAGGGGGCCU | 41 |
| | crRNA | GACGGUAUGAU | |
| SEQ ID NO: 22 | RSV N gene FP | ATACACTATTCAACGTAGTACAGGAGA | 27 |
| SEQ ID NO: 23 | RSV N gene BP | TGAATTTATGATTTGCATCTTCAGTGATT | 29 |
| SEQ ID NO: 24 | RSV N gene | AGATTTCGAAGGTGTGAGGGTGGGAGAGAAA | 61 |
| | Bubble FP | TCTATACACTATTCAACGTAGTACAGGAGA | |
| SEQ ID NO: 25 | RSV N gene | AGATTTCGAAGGTGTGAGGGTGGGAGAGAAA | 63 |
| | Bubble BP | TCTTGAATTTATGATTTGCATCTTCAGTGATT | |
| SEQ ID NO: 26 | RSV N gene- | UAAUUUCUACUAAGUGUAGAUGGAGTGTCA | 41 |
| | crRNA | ATATTATCTCC | |
| SEQ ID NO: 27 | SSFQ-HEX | 5′HEX-TTTTT-3′BHQ1 | 5 |
| SEQ ID NO: 28 | SSFQ-FAM | 5′FAM-TTTTT-3′BHQ1 | 5 |

TABLE 2
Linear RPA primer pool (P45)

| component | Initial conc | Final conc | 20 + Amount(μl) |
|---|---|---|---|
| FP | 100 uM | 10 uM | 10 |
| BP | 100 uM | 10 uM | 10 |
| ddH2O | — | — | 80 |
| Total | — | — | 100 |

TABLE 3
Bubble RPA primer pool (BP45)

| component | Initial conc | Final conc | 20 + Amount(μl) |
|---|---|---|---|
| FP | 100 uM | 10 uM | 10 |
| BP | 100 uM | 10 uM | 10 |
| ddH2O | — | — | 80 |
| Total | — | — | 100 |

Methods for Amplicon-Free CRISPR-Based One-Pot Detection with Loop-Mediated Isothermal Amplification for Point-of-Care Diagnosis of Vital Pathogens

### List of Steps of CoRPLA Assay

In certain embodiments, the 20 to 200 μL of the viral RNA or DNA saliva samples were first treated with lysis buffer including a final concentration 0.2 mM Surfactin (adjusting from 0.1 to 0.5 mM), 50 mM KCl (adjusting from 0 to 50 mM), 0.1 mg/mL SDS, 0.08% Ethanol, 0.05 M, tris-HCL, and 1.5% Tween 20, and then directly loaded to the test tape afterward fabricated with the freeze dried CoRPLA mix (Table 4). For each test, the treated samples were first loaded on the sample pad of the CoRPLA-tape and the tape was then sticked on the wrist. The reaction would start immediately, and color change of the test band is visualized simultaneously. The two-level testing result can be read in 20 minutes. In certain embodiments, a visual readout either by naked eyes or by smartphone camera is observed with the colorimetric reporters modified on the test paper for at most 1 min. For optimal performance of CoRPLA at 30° C., insulation of the test testing strip via clothing coverage or selection of higher-temperature body sites may be warranted in cold environments or for patients exhibiting relatively low skin temperatures.

In certain embodiments, when performing fluorescence readout, a FAM or HEX modified quenched fluorescence reporter (SEQ ID NO: 27-28) can be used, ssFQ-FAM (5′FAM-TTTTT-3′BHQ1) or ssFQ-HEX (5′HEX-TTTTT-3′BHQ1) at a final concentration of 2.5 μM titrated for the best signal-to-noise ratio. In addition, a HEX fluorescence label can be utilized instead of FAM (ssFQ-HEX reporter in Table 1) in an optimization process to check both CRISPR/Cas cleavage activity and amplicon accumulation. If the target is RNA instead of DNA, A final concentration of 1 U/μL Script IV enzyme, 1 U/μL RNase inhibitor, 0.3 mM ATP, 0.1 mg/mL Creatine Kinase and 0.6 μM primer is needed when detecting RNA viral pathogens.

TABLE 4
CoRPLA mix buffer

| Component | Initial conc | Final conc | Amount(ul) |
|---|---|---|---|
| CoRPLA mix buffer | 33.5 | | |
| 10 X Primer pool-P45 | 10 μM | 0.3125 μM | 4 |
| Reaction buffer | 2X | 1X | 13.44 |
| Basic E-mix | 10 X | 1X | 4 |
| dNTPs | 10 μM | 1.8 μM | 7.2 |
| Core Reaction Mix | 20X | 1X | 2 |
| MgOAC (20X) | 280 mM | 20 mM | 2.86--lid |
| LbCas12a mix | 6.5 | | |
| 2.1 NEBuffer | 10X | 1X | 1 |
| LbCas12a | 20 μM | 0.64 μM | 1.28 |
| HPV16 crRNA | 100 μM | 0.64 μM | 0.256 |
| SSFAM | 100 μM | 2.5 μM | 1 |
| glycerol. | 100% | 30% | 3 |
| DNA template | — | — | — |
| Total | | | 50 ul |

*A final concentration of 1 U/uL Script IV enzyme, 1 U/μL RNase inhibitor, 0.3 mM ATP, 0.1 mg/mL Creatine Kinase and 0.6 μM primer is needed when detecting RNA viral pathogens.

The methods of the subject invention relate to testing that using loop mediated isothermal amplification (RPLA) and CRISPR-based signal transduction for fluorescence visual readout, electrochemical-digital readout, or other colorimetric readouts. In certain embodiments, the RPLA forward primers or backward primers can comprise a PAM code downstream from the complementary region of the target sequence of the primer. In other embodiments, the loop structure containing RPLA primer (48-80 base long) can contain an about 20-30-base long sgRNA binding region, an about 5-10 base pair long PAM containing stem region, and/or an about 18-30-base long target binding region. In certain embodiments, the sgRNA binding region, the PAM containing stem region, and the target binding region are adjacent to each other in the RPLA primer.

In certain embodiments, the RPLA forward or backward primers with loop structure is designed with an introduced loop sequence that can be less prone to secondary structure. In some embodiments, the LbCas12a enzyme sgRNA is designed to recognize the complementary chain of the primer binding region after the initiation amplification of the sequence in the target RNA/DNA. In certain embodiments, the introduction of an extra sequence adjacent to the sequence complementary to the target RNA/DNA at 3′ end in the introduced 20-30 base long loop structure at the 5′ end of RPLA primer for CRISPR recognition can solve the sensitivity issue in one-tube reaction by protecting virus RNA/DNA targets in the initial amplification process from cis cleavage by CRISPR/LbCas12a

In certain embodiments, the PAM sequence can be added onto a loop structure containing primers by designing a PAM code in the stem region of a loop structure containing primer, which is about a 7 base pair stem region at both sides of 20-unit long loop structure. This can facilitate the unwinding of the dumbbell structure amplicons to ensure the extension of a DNA polymerase from the DNA 3′ end, such as, for example, Bsu polymerase (New England BioLabs, Ipswich, MA), Twist liquid basic kit (Twist Dx).

In certain embodiments, the binding sequence for sgRNA recognition is in the primer binding region of the RPLA amplicon after dumbbell structure. In certain embodiments, the signal transduction by CRISPR/Cas unspecific collateral cleavage via trans cleavage activity is performed and can last more than 2 hours after activation by cis cleavage activity due to the function of the CRISPR/Cas enzyme towards the activator binding sequence in primer binding region of the RPLA amplicons.

In certain embodiments, the ssFQ reporter is about a 5 base long single stranded DNA sequence in reaction mix buffer with a fluorophore at 3′ end and a quencher at the 5′ end, respectively, preferably with a TTTTT sequence in the middle. In other embodiments, the fluorescence signal is generated from excitation of cleaved fluorophore labelled ssDNA reporters. Further, the simple visual readout is realized by an optical filter on the top of the dark chamber to filtrate the extra lights with a wavelength other than that of the emission light (520 nm to 530 nm) to distinguish positive signal (green color) and negative (dark background color).

In certain embodiments, the CRISPR/Cas recognition sequence can be placed partially on the forward or backward RNA primer, such as about 30 to about 35 nts, by putting a PAM code downstream from the target for CRISPR/Cas recognition of the activator sequence, including at least about 10 nt downstream or upstream from the primer.

In certain embodiments, the high sensitivity of the reaction is achieved by adding 7.5% glycine in the reaction reagent mix without sacrificing amplicon-free performance.

In certain embodiments, the signal is generated from the single-stranded DNA fluorescence reporter (e.g., ssFQ) by separating the fluorophore and quencher to stimulate the fluorescence and generate a fluorescence visual signal. In other embodiments, the signal is generated from double or single-stranded DNA using a methylene blue reporter (dsMB/ssMB) by a differential pulse voltammetry (DPV) measurement to generate an immobilization-free electrochemical based signal. The immobilization-free electrochemical signal can be generated from the difference of diffusion coefficients between the uncleaved and the cleaved methylene blue labeled fragments of the DNA reporter, which can be identified by a differential pulse voltammetry (DPV) measurement using a commercial electrochemical platform.

In certain embodiments, the colorimetric signal is generated from the cleavage of the thiolated single stranded DNA (ssDNA) immobilized on a metal nanoparticle, such as, for example, a gold nanoparticle (AuNP) on surface. The cleavage of ssDNA by CRISPR trans activity leads to aggregation of NPs with the AuNPs plasmonic surface resonance shifts according to the change of the dispersive states of AuNPs. In certain embodiments, the AuNPs plasmonic surface resonance shifts lead to a colorimetric change due to optical properties.

In certain embodiments, the rationally designed dual label at 3′ end and 5′ end of CRISPR trans cleavage reporter and the cleaved and uncleaved dual labeled reporter fragment can be used to lead the colorimetric reporter to different locations on the lateral flow chip in a lateral flow readout. In certain embodiments, there are some capture materials such as Streptavidin for biotin label modified by adsorption on the lateral flow chip test paper surface so that the cleaved reporter with certain colorimetric reporter will be captured at a specific location on the lateral flow chip to enable a colorimetric readout.

In certain embodiments, the rationally designed DNA scaffold of the hydrogel can serve as the 3D sealing layer to coat the colorimetric reporter on to the testing strips, thus the in-situ vertical release of the reporter can take place of its lateral movement to trigger the downstream colorimetric cascade.

In certain embodiments, two-layer DNA hydrogel-based reaction pad enabled real-time colorimetric nucleic acid detection driven by skin temperature. We explored the potential of CoRPLA for visual multi-level diagnostics through a color-changing testing strip using a glucose oxidase (GOx)-triggered colorimetric pad. We examined this concept first in a LFA format with GOx labelled ssDNA linkers. To achieve this, we designed an LFA testing strip with two test bands with different sensitivities for two-level visual testing by adjusting the amounts of the sodium 3,5-dichloro-2-hydroxybenzenesulfonate (DHBS) substrate of the colorimetric reaction in the test bands. Limited by the surface modification of the GOx reporter, the test on the LFA testing strip format required the addition of the washing buffer to flow the GOx to the test band for visual readout. To facilitate the use of the diagnostic testing strip, we further developed a skin adherent-tape, named CoRPLA-tape, via an in-situ 3D DNA hydrogel-sealed colorimetric pad using Cas12a collateral cleavage of ssDNA linkers within the hydrogel.

In certain embodiments, the LbCas12a enzyme sgRNA is designed to recognize the sequence before PAM code in this segment of the target that will become the primer binding region of the RPLA dumbbell structure that facilitates the fast RPLA amplification in the initiation stage.

In certain embodiments, the collected sample input is obtained via whole blood, plasma, serum, lymph, urine, saliva, tears, nasopharyngeal secretions, or any combination thereof. In preferred embodiments, the sample is obtained from nasal secretions or saliva. Once a swap is taken, nucleic acid material, including, for example, the viral RNA can optionally be isolated.

In certain embodiments, the CRISPR recognition sequence can be the pair chain upstream from the conventional RPA primer sequence without a PAM code while it is also necessary for timely regulation of the large amount ssDNA amplicon accumulation in a typical RPA reaction.

### Materials and Methods

### CoRPLA Reaction Buffer Preparation

CoRPLA reaction buffer consisted of Twist RPA basic reaction buffers (20 μL), dNTPs (1.8 mM), MgOAc (20 mM), Creatine Kinase (0.1 μg/μL), ATP (3 mM), and primers (0.3125 mM). For RNA templates, Rtx Reverse Transcriptase (300 units/μL), and Proteinase inhibitor (1×) were added. Buffer was prepared as 30 μL aliquots and then stored at −20° C. for up to 1 month.

### CRISPR/LbCas12a Mix and Cleavage Reaction

The assembled LbCas12a-sgRNA complex (0.64 μM) was incubated with 2.5 μM ssDNA reporters in 2.1 NEB buffer (pH 8.0) at room temperature for 15 min. 100% glycerol was then added to enhance the reaction sensitivity before storing the mix in 3.5 μL aliquots (0.5 μL reaction, 3 μL glycerol) at −20° C. for up to 6 months. The CRISPR mix, CoRPLA reaction buffer, and analyte template were mixed to a final volume of 40 μL to initiate the cleavage reaction.

### CoRPLA Primer Selection

We designed primers using a two-round selection process. In the first round, primers were designed using the NCBI primer design tool and evaluated for amplification efficiency using qPCR, amplification melting curve analysis, and fragment-based selection (see below). In the second round, we added strings of 2-3 A/Ts at the 5′ end to facilitate target unwinding and 2-3 G/C at 3′ end to improve extension efficiency and compare the efficiency in RPA.

### Bubble Primer Design

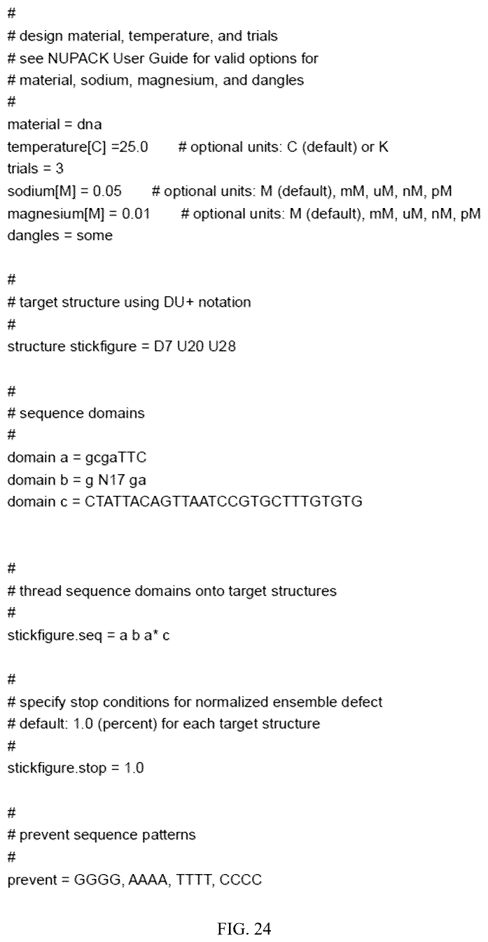

Numerical simulation method of loop structure is shown in FIG. 24. The design was done on NUPACK.org²⁵.

### Chemicals

DNA oligonucleotides and sgRNA were either purchased from Integrated DNA Technologies (IDT) or Generay (Shanghai, China). LbCas12a was purchased from MEGIGEN (Guangzhou, China). dNTPs, MgOAc, Q5 polymerase, Bst 2.0 WarmStart DNA Polymerase, AMV Reverse Transcriptase, and proteinase K inhibitor were purchased from New England Biolabs (NEB). SuperScript™ IV Reverse Transcriptase was purchased from Thermo Fisher Scientific. Creatine Kinase and ATPs were purchased from Yeason (Shanghai, China). Twist RPA basic reaction buffers were purchased from TwistDx (United Kingdom). N,N,N′,N′-tetramethylethylenediamine, NaCl, KCl, MgCl₂, and ATP lyophilized powder were purchased from Sigma. Acrylamide/bisacrylamide solution was purchased from Bio-Rad. SYBR Gold Nucleic Acid Stain, SYBR Green Nucleic Acid Gel Stain, SYTO 16 Green Fluorescent Nucleic Acid Stain, 1 M Tris-HCl, pH 8.0, and RNAse-Free Distilled Water were purchased from Thermo Fisher Scientific. Glucose, horseradish peroxidase (HRP), glucose oxidase (GOx), 4-aminoantipyrine (4-AAP), and sodium 3,5-dichloro-2-hydroxybenzenesulfonate (DHBS) were obtained from Merck.

Immobilization of Colorimetric Reagents onto the Lateral Flow Testing Strip

Anti-biotin antibodies were absorbed onto the surface, which was then blocked with bovine serum albumin (BSA). Then, linker DNA modified with biotin and FAM labels was incubated. Finally, a GOx tagged to an anti-FAM antibody was immobilized. All the reagents are modified by adsorption at different incubation times. The first antibody layer (anti-biotin antibody) and BSA layer are incubated for 1 h each in series. Then, the reporter DNA layer (Biotin-FAM reporter), biotin, and GOx antibody layer is incubated for 15 min each in series. After each incubation step, the substrate was rinsed with wash buffer (50 μL of 0.05% TWEEN 20 in 10 mM PBS). RPA and CRISPR/Cas detection reagents were deposited on the conjugate pad and air dried. Nitrogen flow was then used to remove the humidity. Colorimetric reaction reagents (see below) were similarly deposited on nitrocellulose membranes and air dried. DNA hydrogel is then coated on the substrate (see in next section: DNA hydrogel fabrication). Tests bands were taped to the test testing strip using water-proof skin-like medical tape. Throughout the process, membranes of different pore sizes were evaluated. Testing strips were modified with poly-methylsilsesquioxane (MSQ) to create a hydrophobic zone between each test band and reduce signal leakage due to chromophore leaching.

Fabrication of substrates layer (Table 5): A nitrocellulose membrane (0.2 μm pore-size) was chosen as the substrate for sensing reagents including chromogenic dyes, horseradish peroxidase (HRP) and oxidases. The sensing reagents for glucose oxidase (GOx) included 8 mM 4-AAP in DHBS buffer, 1 mg/mL HRP and 200 mM glucose in DI water. For GOx detection, quadrants were firstly modified with 1.25 μL of chromogenic dye solution and 1 μL glucose solution. After drying, 0.5 μL of HRP solution was uniformly drop-cast onto the respective sensing quadrants on the nitrocellulose membrane, followed by another addition of 1.25 μL dye solution. A certain amount of glucose oxidases (0-10 μg) was added as input in multi-level visual readout optimization work. The nitrocellulose membrane was cut out into 2 mm-wide thin testing strip ready for lateral flow testing strip integration covered with skin-adherent transparent medical tape.

TABLE 5
Colorimetric substrate layer modification protocol.

| Layers | | Stock conc | Final conc | | Volume |
|---|---|---|---|---|---|
| 1 | 4-AAP in 1 mM | 16.26 mg/1 mL = 2X | 4 | mM | 1.25 |
| | DHBS | | | | |
| 2 | HRP | 1 mg/ml = 10X | 0.1 | ug/uL | 0.5 |
| 2 | Glucose | 18.0156 mg/0.5 mL = 5X | 40 | mM | 1 |
| 3 | 4-AAP in mM | 16.26 mg/1 mL = 2X | 4 | mM | 1.25 |
| | DHBS | | | | |
| 4 | GOD | 5 mg/0.5 ml = 10X | 1 | ug/uL | — |

| | | | | |
|---|---|---|---|---|
| 4 | DI | — | — | — |

### DNA Hydrogel Fabrication

The scaffold (Y) and linkers (L) were first created separately. Component oligos (Y1, Y2, Y3: 200 μM; L1 L2, 200 μM) were annealed for 3 hours in a 0.5× dilution of hydrogel buffer (20 mM Tris-HCl, 50 mM NaCl, 5 mM KCl, 10 mM MgCl2, pH 7.5). The hydrogel buffer was then evaporated using the vacuum concentrator for 80 mins at 30° C. or concentration columns at room temperature. The annealed scaffold (0.4 mM) and linkers (0.6 mM) were then resuspended together in 1× hydrogel buffer to ensure cross-linking before crystallization. 0.05% polyvinyl alcohol (PVA), 1×SYBR Green, and 10 mg GOx were added at before 3-hours gelation on the nitrocellulose membrane at 37° C. in a metal block incubator. 30 μL Rnase-free H2O was used to wash out free reporter (GOx/AuNPs).

### Image Mask Processing

We used the edge-finding function in ImageJ to create a rough mask in the gray-color channel. We then used the color merge function to create a mask tainting image preprepared for the deep learning process. While the original figure kept all color channel expect gray-color, the mask filled in the gray-color channel. To complement the training dataset, accurately and manually classified feature images were rotated and mirrored, resulting in a total of 1000 images. Of these images, 75% were used for training and 25% were reserved for validation. The training data for each epoch were fed into the network in random order to ensure generalizability. To prevent overtraining, the training was stopped after classification accuracy on the validation image set ceased.

### Deep Learning-Based Band Recognition

The support image and query image first encode high-dimensional features and share weights. The support feature is then divided into multiple overlapping meshes. Each grid uses masked average pooling to extract support prototypes from support features. Finally, the calculation supports grid-based cosine similarity between prototype and query features to achieve good band segmentation.

Of these images, 75% are used for training datasets and 25% are treated validation datasets. In addition to the backbone and feature fusion network, the FPN layer was used to up-sample the output feature map generated by multiple convolutions down-sampling operations from the feature extraction network. Multiple new feature maps were thus generated to detect different-scale targets.

After the three feature maps were sent to the prediction head, the confidence calculation and bounding-box regression were executed for each pixel in the feature map using the preset prior anchor. This generated a multi-dimensional array (Bboxes) that includes object class, class confidence, box coordinates, width, and height information. The corresponding thresholds were then set to filter the useless information in the array, and a non-maximum suppression (NMS) process was performed to output the final detection information.

YOLOv5 returned three outputs: the classes of the detected objects, their bounding boxes, and the object scores. To compute the classes loss and object loss, the algorithm used BCE (Binary Cross Entropy). On the other hand, CioU (Complete Intersection over Union) loss was used to compute the location loss.

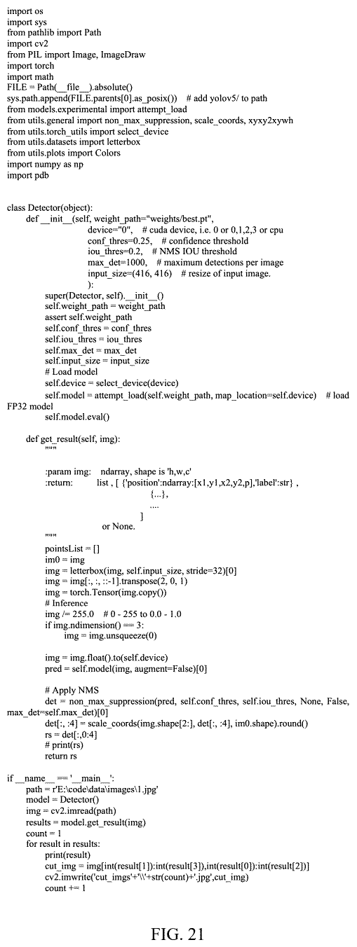

The related model code was released in GitHub (see worldwide website: github.com/yzhueh/BRRS), the BRRS main function is shown in FIG. 21.

### Machine Learning-Based Semi-Quantitative Analysis

Random forest model and the agglomerative clustering method were used to achieve semi-quantitative analysis.

Random forest, a method suitable for high-dimensional datasets and robust to outliers and missing data and, was also used for both classification and regression tasks. Numerous decision trees were each independently trained on a random subset of the training data and a random subset of the features. The final prediction was then made by combining the predictions of all the decision trees. This approach reduces the risk of overfitting and improves the accuracy of the algorithm.

Agglomerative clustering starts by treating each data point as a singleton cluster and then iteratively merges the closest clusters based on a similarity measure, forming a hierarchy of clusters. This bottom-up approach allows for building the hierarchy from the individual elements by progressively merging clusters.

The related model code was released in GitHub (see worldwide website: github.com/yzhueh/BRRS).

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLES

A greater understanding of the present invention and of its many advantages may be had from the following examples, given by way of illustration. The following examples are illustrative of some of the methods, applications, embodiments, and variants of the present invention. They are not to be considered as limiting the invention. Numerous changes and modifications can be made with respect to the invention.

### Example 1—Loop Mediated Amplification Accelerates the Activation of CRISPR/CAS and Enhances Signal Transduction Efficiency

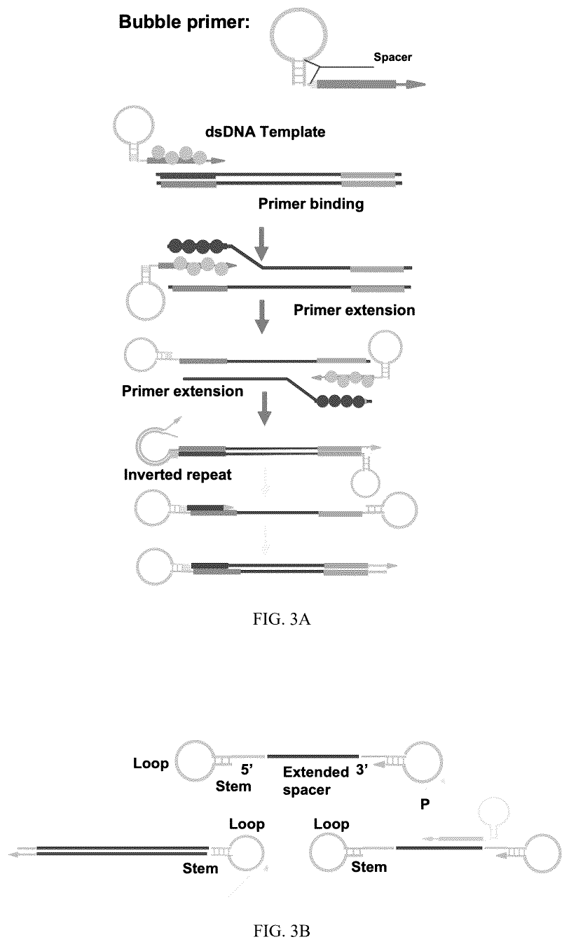

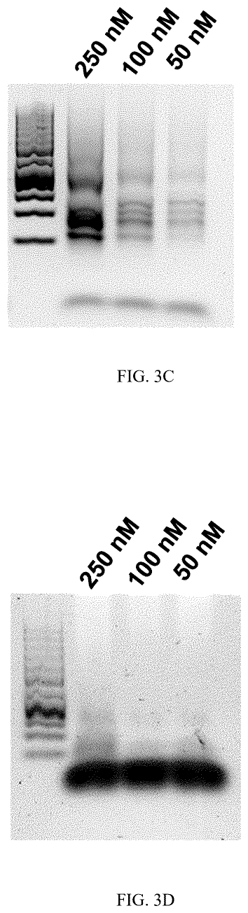

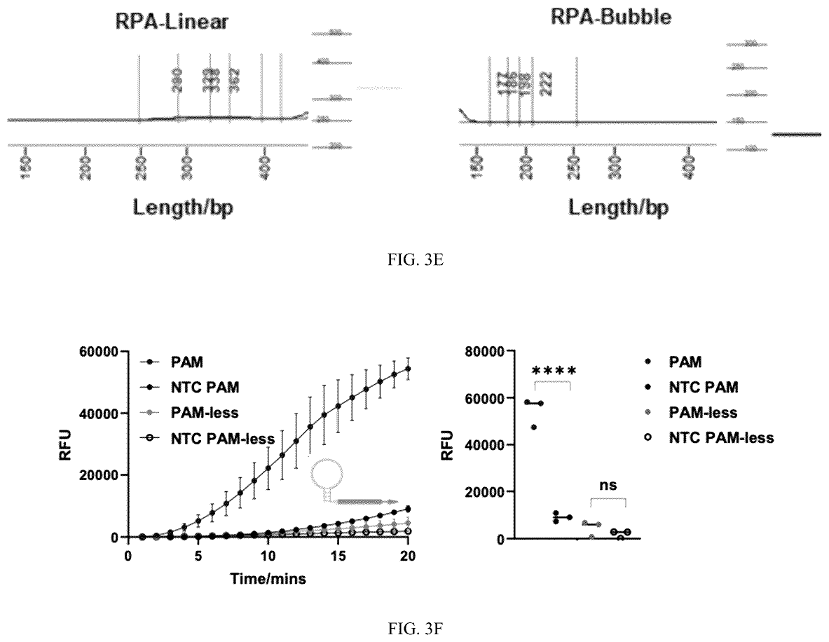

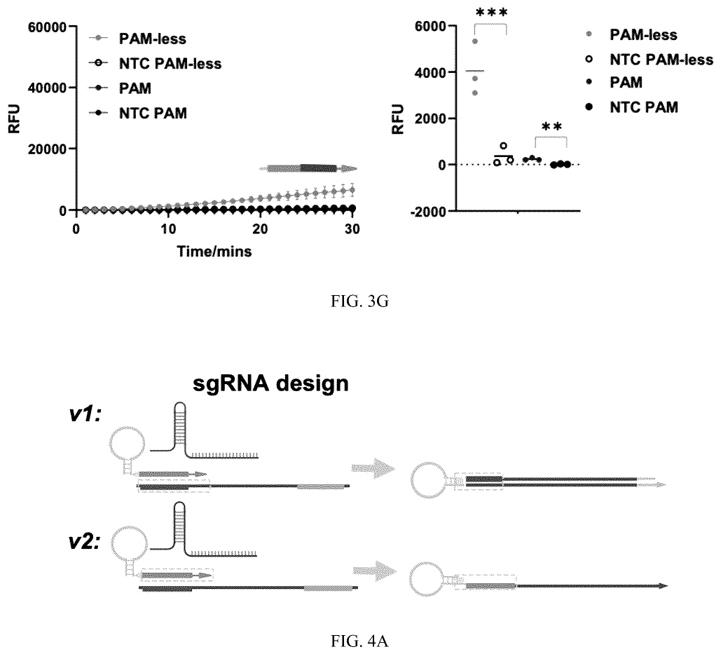

In each reaction, to accelerate the kinetics of RPA, we designed a loop-mediated process using a bubble primer. In FIG. 3A, the bubble primer binds with dsDNA templates, assisted by the recombinase and single-strand DNA-binding proteins (SSB), to generate a LAMP-like loop-containing amplicon via exponential amplification with the forward and reverse primers like in traditional RPA. Additionally, the loop-containing amplicon can self-fold and extend itself using its own loop, which accelerates the DNA amplification process. To test this design, we first introduced the looped structure to the intermediate template to verify the loop-mediated amplification at a low temperature (FIG. 3B). This system induced continuous amplification, generating a LAMP-like product profile at 37° C. without recombinase and SSB in the system (FIG. 3C). When a looped primer was used for this loop-mediated self-amplification, the LAMP-like product profile was still observed, although with a decreased amplicon amount (FIG. 3D). The fragment analysis of RPA with the bubble primer showed a similar profile to the LAMP-like self-induced amplification with the looped primer, where the major products had relatively low molecular weight (FIG. 3E). These results suggest that the LAMP-like self-induced amplification introduced by the bubble primer design can occur at 37° C. and may contribute to the overall amplification kinetics alongside the recombinase-induced conventional RPA.

A more effective CRISPR-RPA system could be developed by combining our faster RPA process and an optimized CRISPR system, exploiting the strengths of both technologies. To test this, we compared the performance of two primer designs: a traditional linear primer and our novel bubble primer, within a one-pot reaction. After primer sequence optimization, we observed that the bubble primer RPA with the CRISPR/Cas12a system with optimal PAM yielded a significantly higher signal with the selected primer (FIG. 3F), while the linear primer showed no significant signal variation (FIG. 3G). Although the linear primer produced a slight signal response with the PAM-less CRISPR system, the bubble primer resulted in a ten-fold increase in end-point fluorescence intensity compared to the linear primer. Based on these results, we hypothesize that the bubble primer RPA produces more dsDNA than ssDNA amplicons, which differs from other one-pot Cas12a/RPA reactions where a significant amount of ssDNA is produced^{22,26,27}.

The inclusion of the PAM sequence within the bubble primers led to a 5-fold increase in signal fluorescence compared to the PAM-less design due to the quicker CRISPR/Cas activation by PAM-containing activators during the amplification initiation stage (FIG. 3F). The novel bubble primer design leverages the efficiency of both the inverted ‘LAMP-like’ amplification products and the PAM-dependent Cas12 activation, while also eliminating the need for a PAM-containing target sequence.

### Example 2—Optimization of CORPLA SGRNA Recognization Region to Enhance High Sensitivity for the Detection at 30° C.

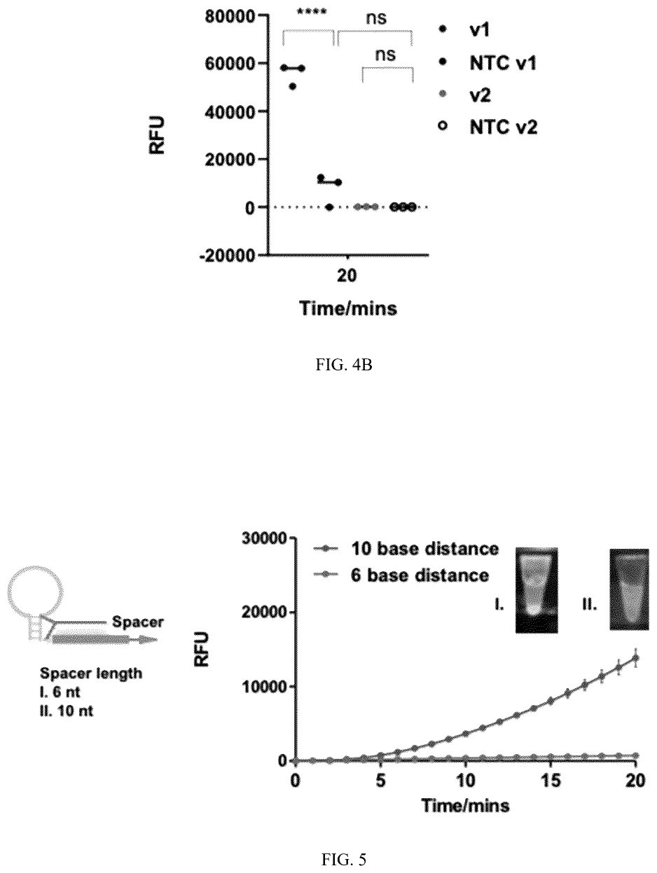

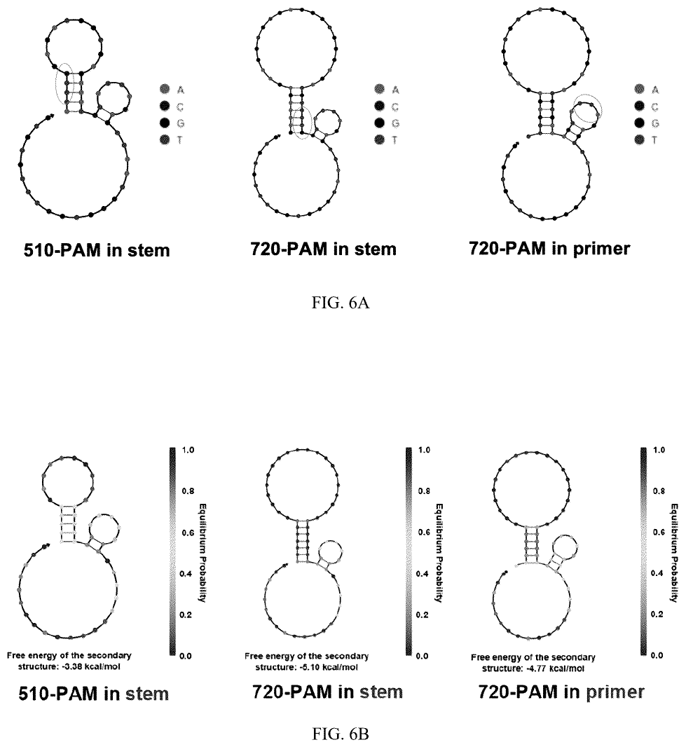

Different sgRNA and activator designs lead to different signal generation and amplicon-depleting performance by CRISPR/Cas12 cis-cleavage. For this, we tested two versions of sgRNA using the bubble primer (FIG. 4A). In version 1 (v1), the sgRNA targets the complementary sequence of the forward primer (i.e., the same strand of the ssDNA template), while in version 2 (v2), the sgRNA recognizes the strand extended from 3′ of the forward primer (i.e., the complementary strand of the ssDNA template). We observed a higher fold change in the signal with the v1 design, whereas v2 showed no significant difference between the positive test and its non-target control (NTC), and the two NTC tests showed no obvious signal amplification (FIG. 4B). It is worth noting that, for the sgRNA v1 design, there is no activator sequence in the primer, which would only be present in the fully amplified dsDNA amplicons forming the inverted repeat structure by polymerase extension, similarly to that in conventional LAMP 28. The loop structure in the bubble primers also impacts the binding of Cas12 by steric hindrance until it is opened by the primer extension by the reverse primers forming the inverted repeats. Thus, the PAM-containing bubble primer coupled with the v1 sgRNA design effectively keeps Cas12 inactive until primer extension. Additionally, a shorter spacer length between the stem and the PAM region in the bubble primer further reduced the background signal (FIG. 5). This may be due to the hairpin-loop structure efficiently blocking the PAM and activator region from Cas12 by steric hindrance, specifically when the PAM region is located closer to the stem. Secondary structure predictions suggest that the hairpin loop remains relatively stable at 30° C., supporting the rationale behind our design (FIG. 6B).

### Example 3-Optimization of CORPLA Buffer to Achieve Good Sensitivity and Specificity with Amplicon-Depleted Performance at 30° C.

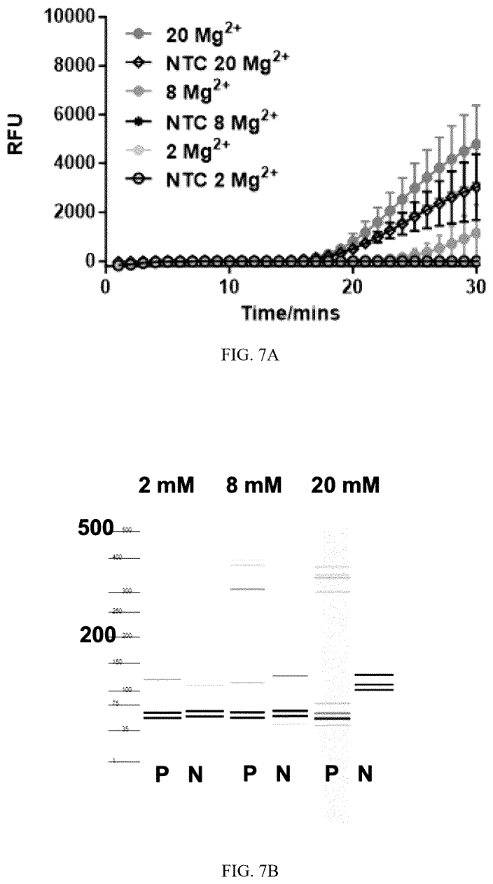

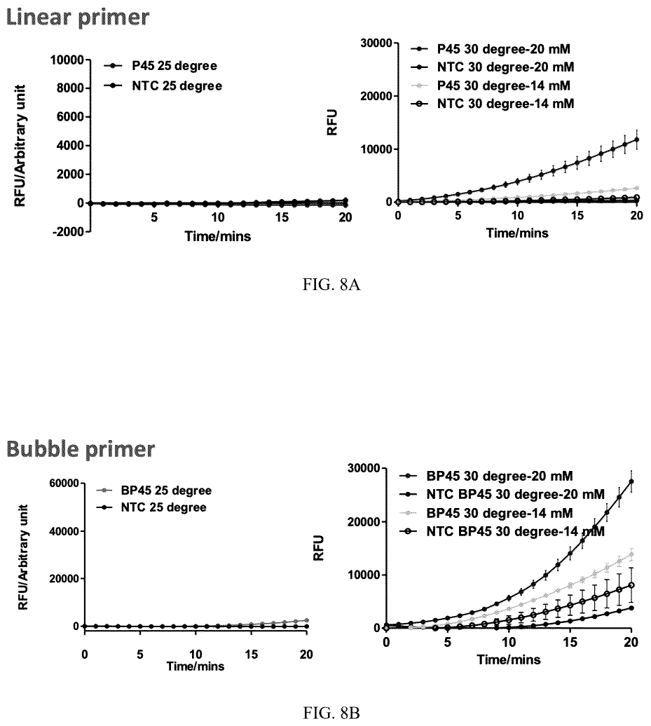

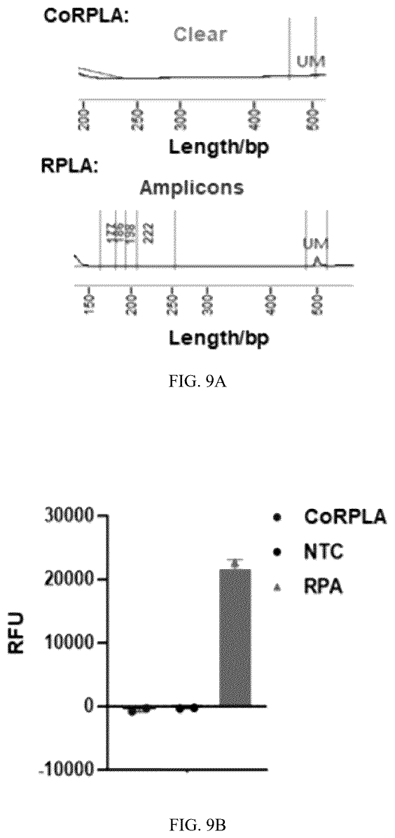

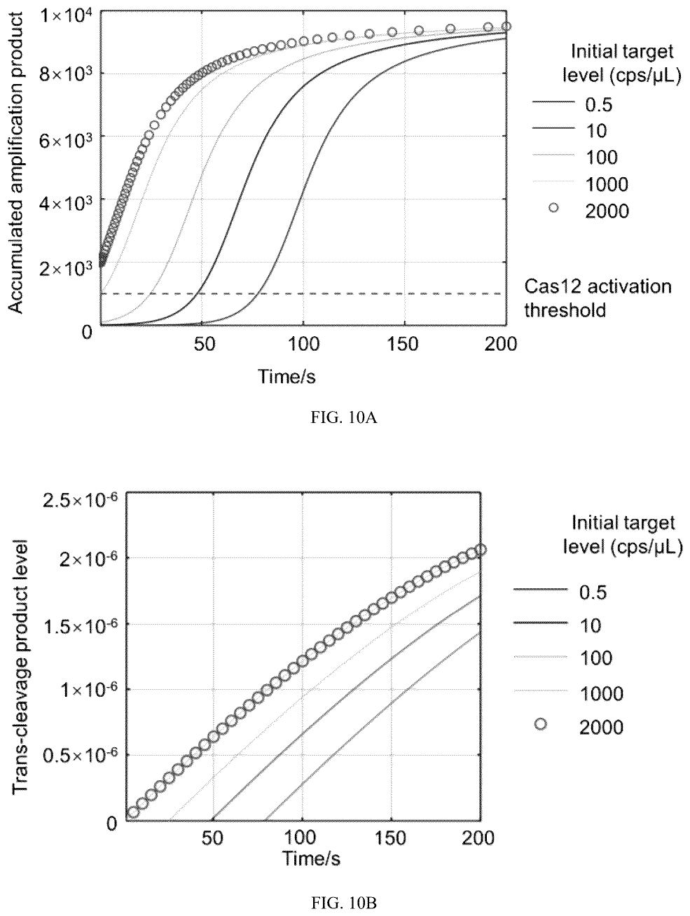

To perform CoRPLA detection at skin temperature, which is around 30° C.²⁹, the bubble primer design with the v1 sgRNA design was chosen for its superior signal amplification efficiency. Although the cleavage activity of LbCas12a cleavage activity is generally better at 30° C. than at 37° C.^{30,31}, RPA sensitivity is impacted at 30° C. due to a 50% reduction in polymerase activity and a significantly reduced single-strand binding protein activity^{27,32}. We therefore increased the concentration of magnesium acetate (MgOAc) to enhance polymerase binding to primers for quicker initiation kinetics and reduced secondary structure formation³³. We first examined the impact of MgOAc concentration on RPA amplification. Higher MgOAc concentrations enhanced the efficiency of target specific and non-specific RPA at 30° C. (FIGS. 7A and 7B). By increasing the MgOAc concentration in CoRPLA reaction at 30° C., the results showed an improvement in Cas12-induced signal generation (FIGS. 8A and 8B) and a reduction in non-specific signals at 20 mM MgAOc, while the amplicon depletion property of CoRPLA was retained (FIG. 9A). The reduction in non-specific signals may be related to the enhanced target recognition specificity of Cas12 enzymes with increased of Mg²⁺ concentrations^{34,35}. Additionally, extra ATP and creatine kinase were added to preserve the protein structure when extra reverse transcriptase is included for detecting RNA targets³⁶.

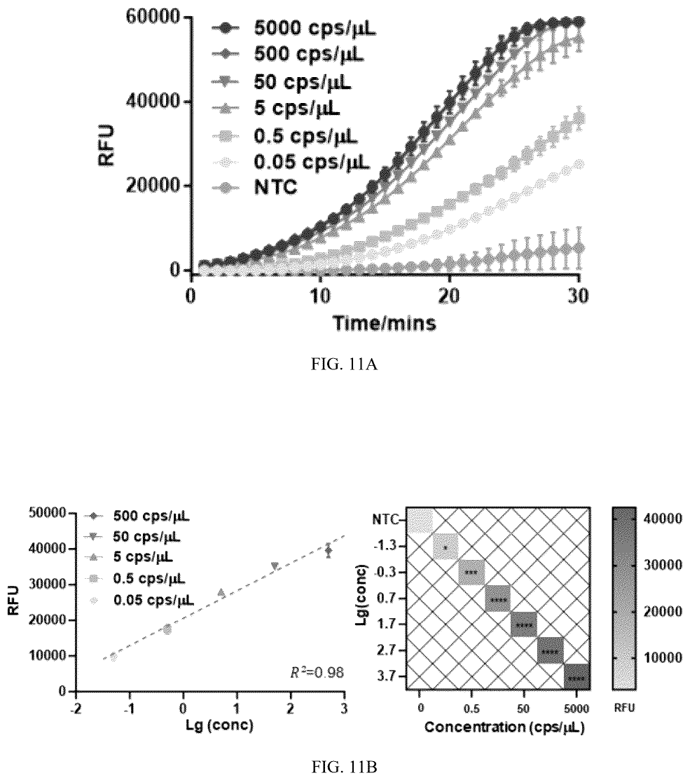

Using the optimized reaction, CoRPLA efficiently detected HPV cfDNA at concentrations as low as 0.5 cps/μL at 30° C. while showing effective amplicon depletion performance (FIG. 9B), benefited from the CRISPR-based kinetic regulation of the exponential amplification (FIGS. 10A and 10B).

### Example 4—the Timely Regulation of CRISPR/CAS in CORPLA Assay can Achieve Semi-Quantitative Detection at 30° C.

The kinetic profile of the signal generation of CoRPLA, is nearly linear before the reaction plateaus (FIGS. 11A and 11B). We hypothesized that this is due to the signal amplification being primarily driven by the trans-cleavage activity of Cas12, which can be described by the Michaelis-Menten equation 37.38 In our system, the activation of Cas12 depletes the generated amplicons and suppresses the RPA reaction. Assuming that no signal is generated until the activation of Cas12 and that the activated Cas12 trans-cleavage follows the same kinetics, we modelled the CoRPLA signal generation using a recently reported RPA kinetic model³⁹ and the Michaelis-Menten equation (FIG. 10A). The model suggested that the end-point signal of CoRPLA could be dose-responsive within a certain range of initial target concentrations (i.e., target level below the activation threshold of Cas12). As the model predicted, the response of CoRPLA to different concentrations of nucleic acid targets using fluorescence successfully differentiated input target levels ranging from 0.5 cps/μL to 500 cps/μL (FIG. 11A), though the results also displayed kinetics of early trans-cleavage initiation and nucleic acid amplification suppression not considered in our simplified model (FIG. 10B). The end-point signals measured at 20 minutes were proportional to the initial target concentration with a R2 value of 0.98 in a linear regression (FIG. 11B). These findings demonstrate the semi-quantitative capability of CoRPLA through a dose-responsive end-point signal, highlighting its potential for development into a colorimetric-based visual multi-level testing method.

### Example 5—the CORPLA Assay for RNA Detection at 30° C.

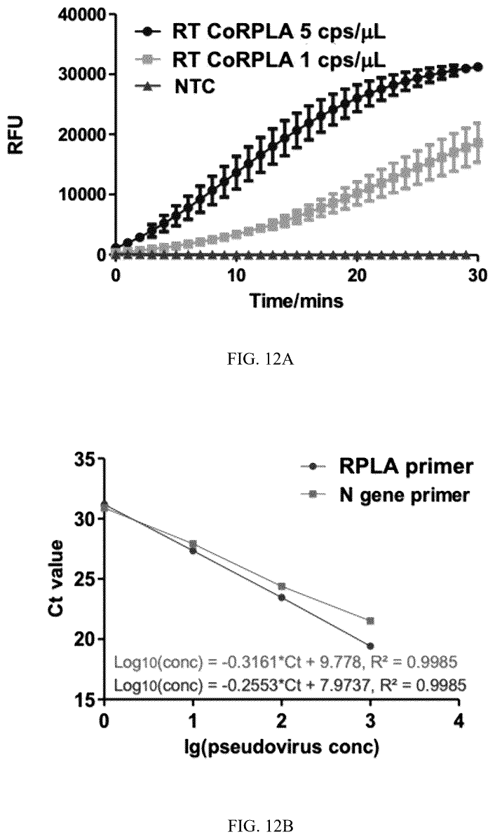

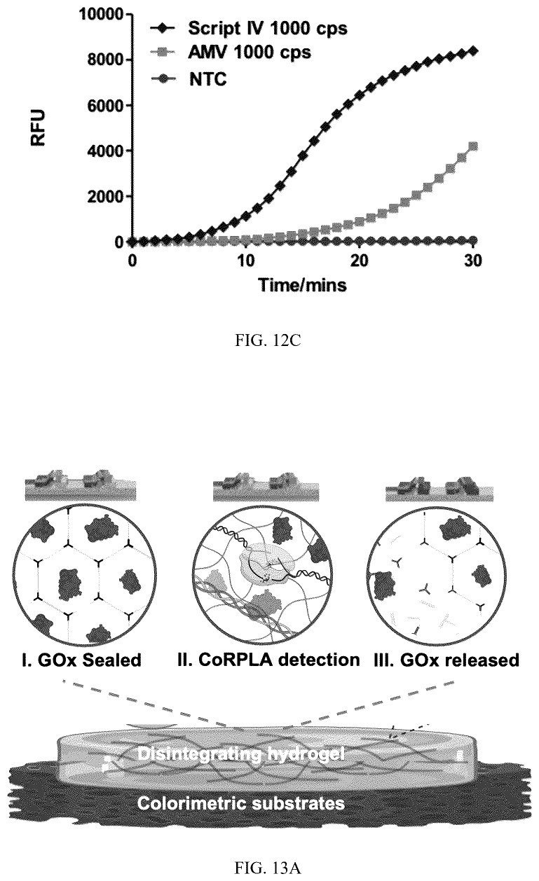

When combined with reverse transcriptase, the CoRPLA assay (RT-CoRPLA) also detected SARS-CoV-2 viral RNA at 30° C. at concentrations as low as 1 cp/μL, showing the feasibility of CoRPLA as a deployable method for detecting various pathogens (FIGS. 12A, 12B, and 12C).

### Example 6-Two-Layer DNA Hydrogel-Based Reaction Pad Enabled Real-Time Colorimetric Nucleic Acid Detection Driven by Skin Temperature

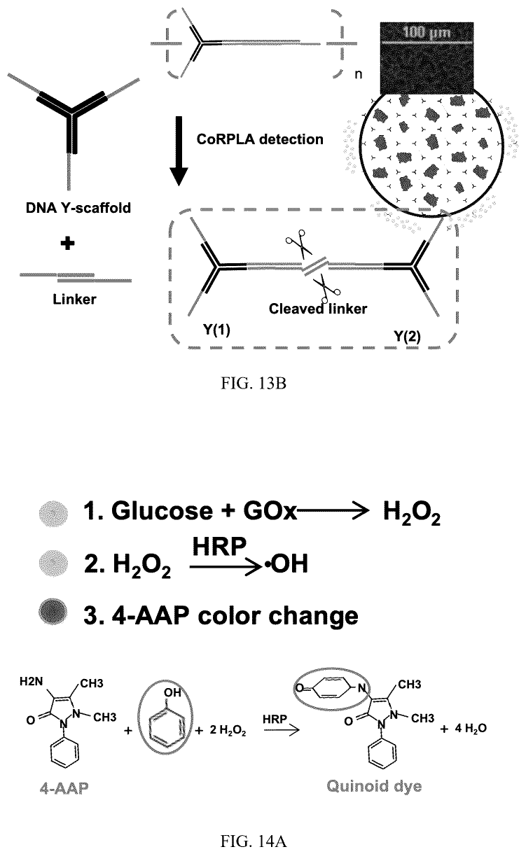

Limited by the surface modification of the GOx reporter, the test on the testing strip format required the addition of the washing buffer to flow the GOx to the test band for visual readout. To facilitate the use of the diagnostic strip, we developed a skin adherent-tape, named CoRPLA-tape, via an in-situ 3D DNA hydrogel-sealed colorimetric pad using Cas12a collateral cleavage of ssDNA linkers within the hydrogel⁴⁰. The design eliminates the need for manual operation after sampling, as the simultaneous colorimetric readout is enabled by the in-situ release of the GOx reporter upon the trans-cleavage of the DNA hydrogel scaffold during the CoRPLA reaction⁴⁰. The colorimetric pad in which the GOx is sealed in the DNA hydrogel is directly assembled on top of a substrate layer containing the colorimetric readout reagents and the CoRPLA reagents in CoRPLA-tape as the test bands (FIG. 13A). The DNA scaffold within the hydrogel was assembled from double-stranded ‘Y’-motifs, which constituted the vertices of the scaffold, and single-stranded ‘linker’-motifs, which constituted the edges (FIG. 13B), based on a previous DNA hydrogel design⁴¹. When the analyte is present, the DNA scaffold is destroyed by the trans-cleavage of the linker motifs within the hydrogel, releasing GOx into the nitrocellulose (NC) layer beneath and ultimately triggering the colorimetric cascade. As before, the test bands containing varying concentrations of DHBS with different detection sensitivities are included in each test strip for multi-level testing.

### Example 7—Colorimetric Cascede Triggered Tunable Multi-Level Visual Readout on Testing Strip

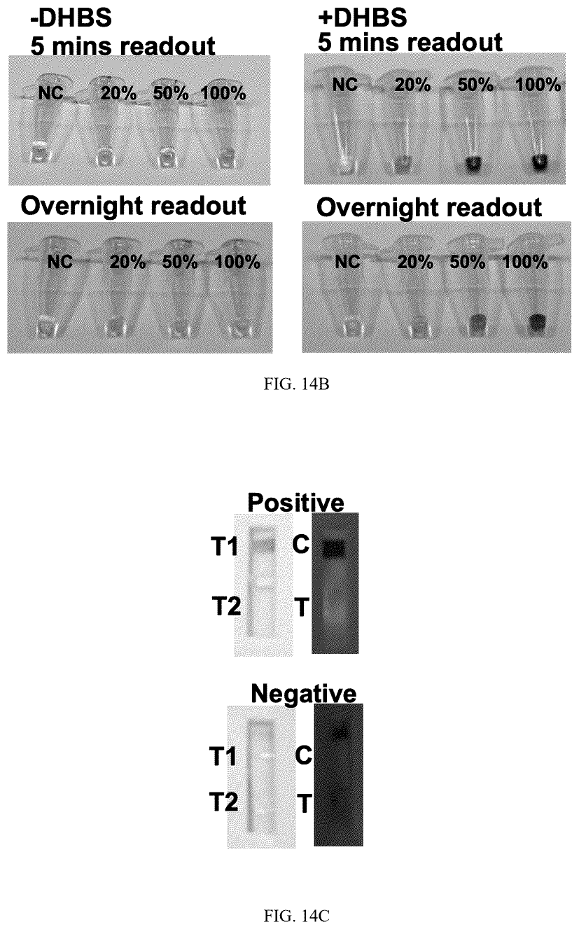

We further explored the potential of CoRPLA for visual multi-level diagnostics through a color-changing strip using a glucose oxidase (GOx)-triggered colorimetric pad. We examined this concept first in a LFA format with GOx labelled ssDNA linkers. To achieve this, we designed an LFA strip with two test bands with different sensitivities for two-level visual testing by adjusting the amounts of the sodium 3,5-dichloro-2-hydroxybenzenesulfonate (DHBS) substrate of the colorimetric reaction in the test bands (FIGS. 14A, 14B, and 14C).

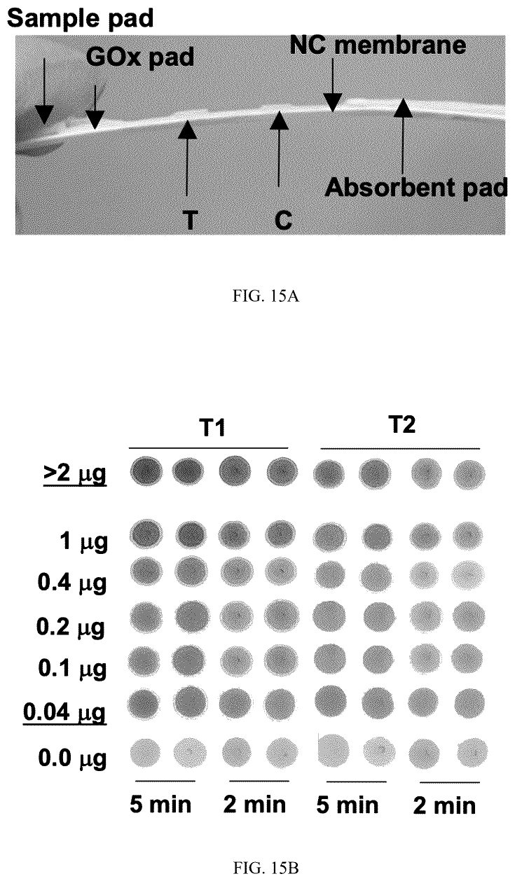

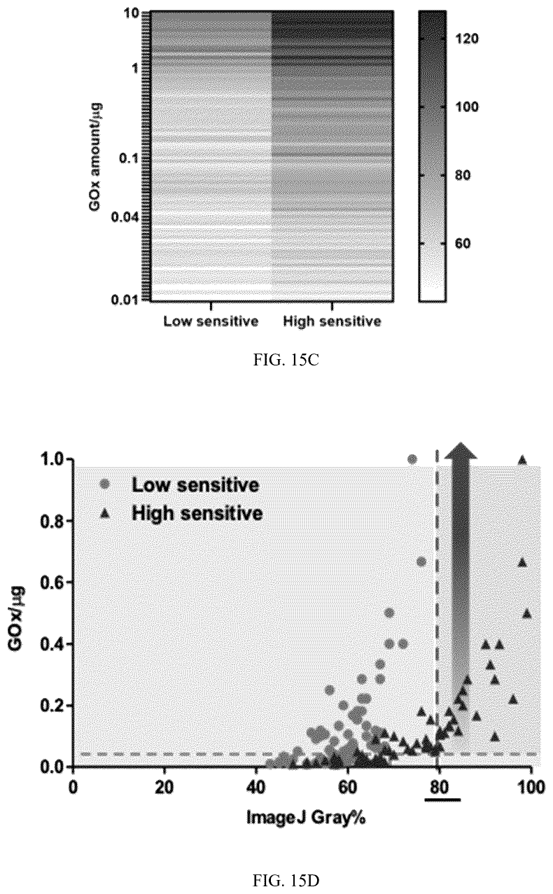

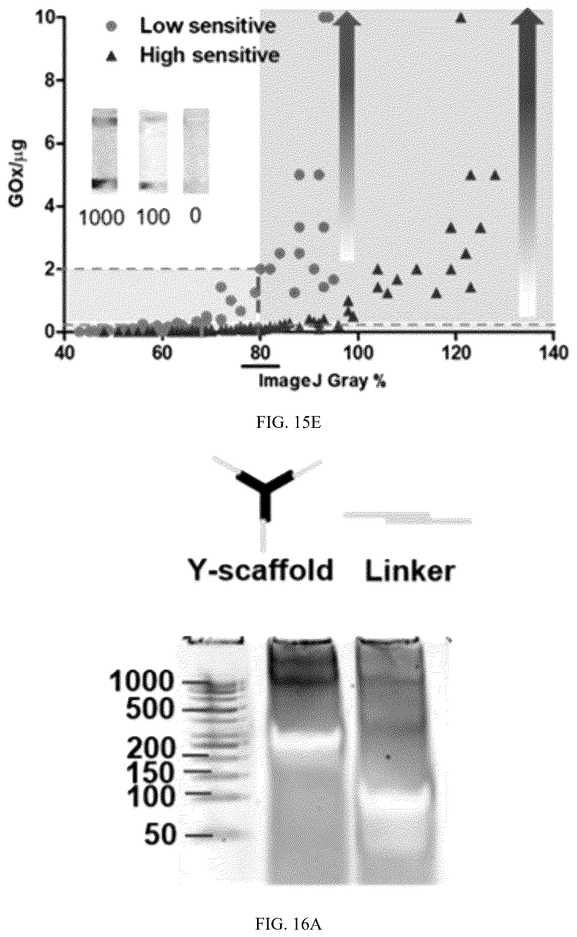

After optimization on the GOx ssDNA probe length for best signal-to-noise ratio (adjustable from 5 to 15 nt length), a LFA strip with two test bands with sensitivities of 1000 cps/μL and 0.5 cps/μL respectively, enabled two-level testing with visual readout that distinguishes between negative, low viral load (0.5-1000 cps/μL), and high viral load (over 1000 cps/μL) samples (FIGS. 15A, 15B, 15C, 15D, and 15E).

### Example 8—Optimization of the DNA Hydrogel Coated Test Band on Wearable Strips

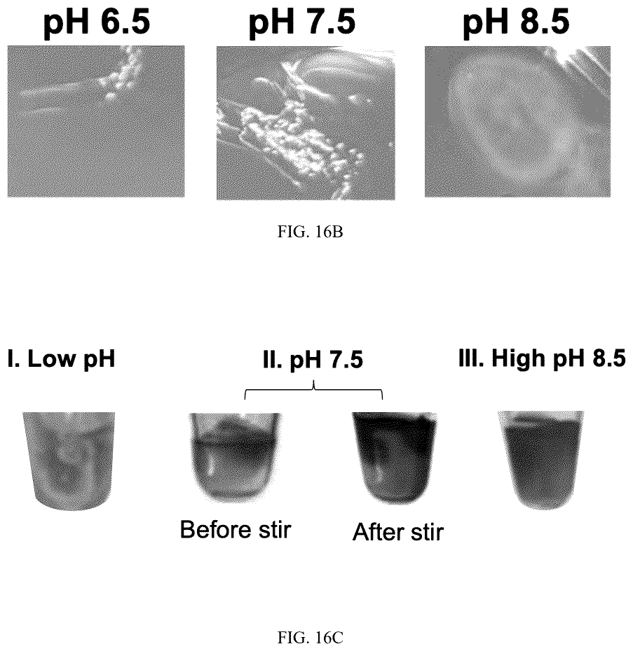

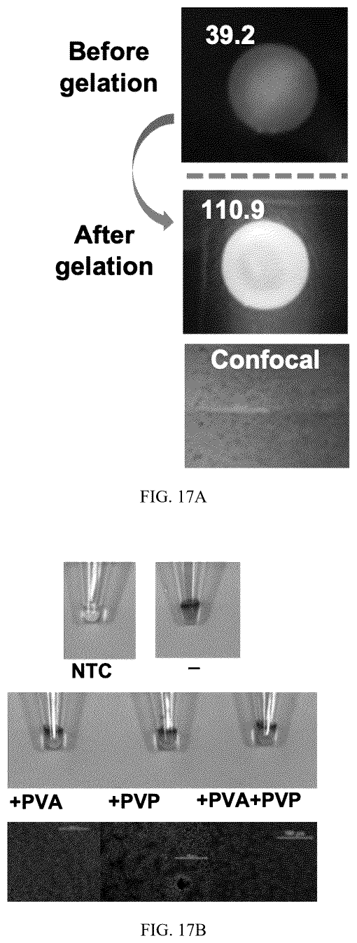

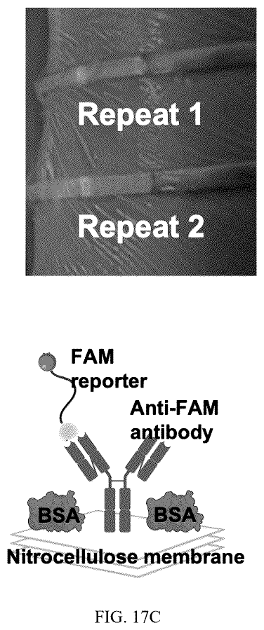

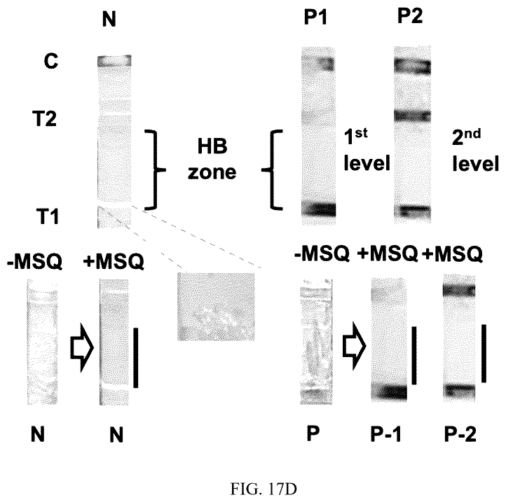

The development of the DNA hydrogel test bands involved optimization of gelation condition and additives to achieve best pore-size with strong mechanical properties. We determined that a pH of 7.0-7.5 is necessary to maintain a stable dsDNA scaffold (FIGS. 16A, 16B, and 16C). At low pH, protonation of the negatively charged phosphate groups in the DNA phosphodiester backbone leads to structural changes and reduced stability of the DNA molecule⁴¹, resulting in low-degree gelation and a dispersed hydrogel. Conversely, high pH destroys the base-pair interactions and causes the dsDNA to unwind⁴². The band position and intensity in gel-based assessment of annealing validation demonstrate the formation and stability of Y-shaped DNA scaffolds and L-shaped linkers, which can be used for the construction of the DNA hydrogel (FIG. 16A). To prevent GOx leakage in negative assays, we optimized the gelation efficiency by using the intercalating dye SYBR Green to image the dsDNA skeleton and quantitatively characterize the degree of gelation under a confocal microscope (FIG. 17A). We evaluated the hydrogels of different pore sizes (5-100 μM) using several gelation additives at different concentrations for the better control of GOx leakage and the reinforced mechanical strength than pure DNA. Ultimately, we selected 0.05% polyvinyl alcohol (PVA) to create a pore size of 5 μm for enhanced GOx encapsulation with reduced leakage and good release efficiency after reaction activation (FIG. 17B).

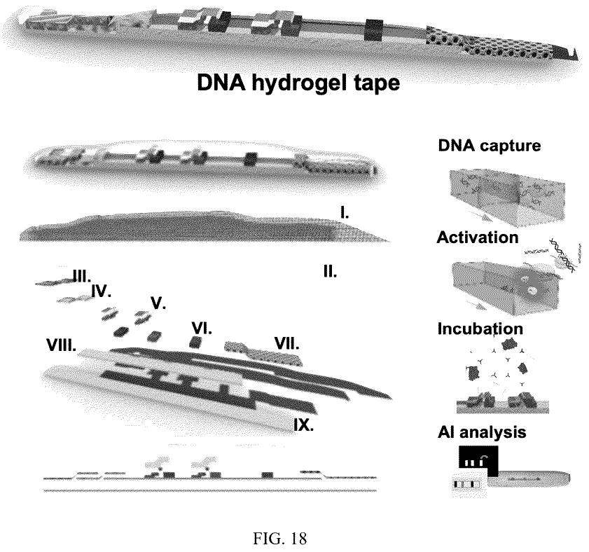

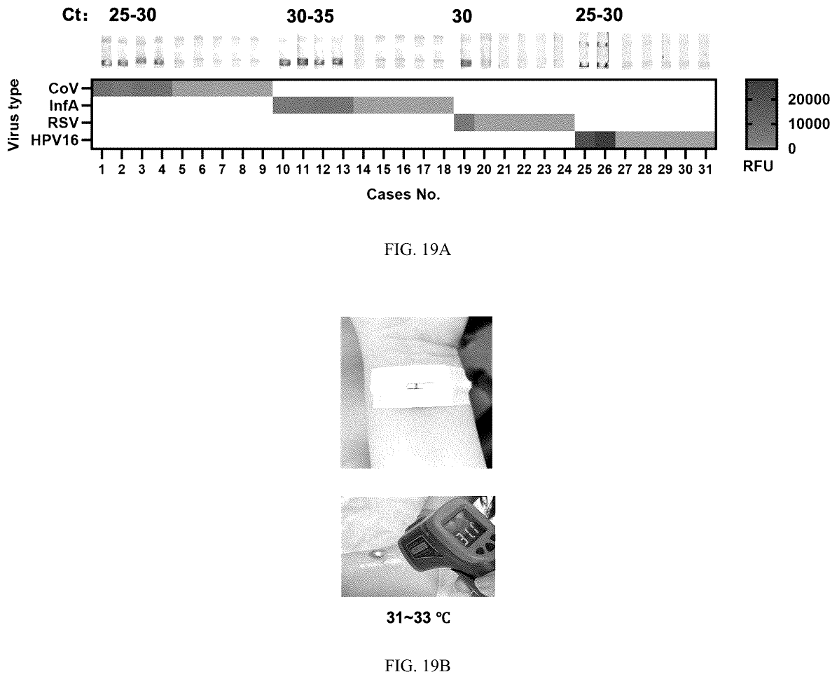

After the validation of the skin-temperature CoRPLA via a fluorescence assay (FIG. 17C). However, decreased flow stability during on-skin tests may cause the test band boundaries to appear blurrier than in a normal standalone lateral-flow on the strip, we modified the strips with poly-methylsilsesquioxane (MSQ) to create a hydrophobic zone between each test band, reducing signal leakage caused by leaching from the test bands (FIG. 17D). With these improvements, we achieved clear multi-level testing using the CoRPLA assay in an on-skin, wearable tape format (FIGS. 18, 19A, and 19B).

### Example 9—Mobile Phone-Assisted Multi-Level Diagnostics Using CORPLA Testing Strip and Machine Learning Interpretation

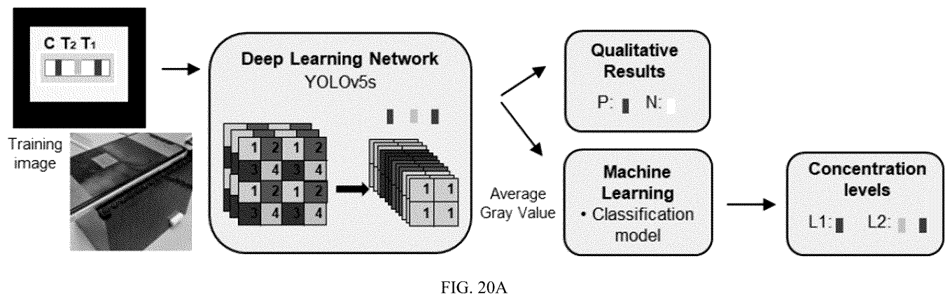

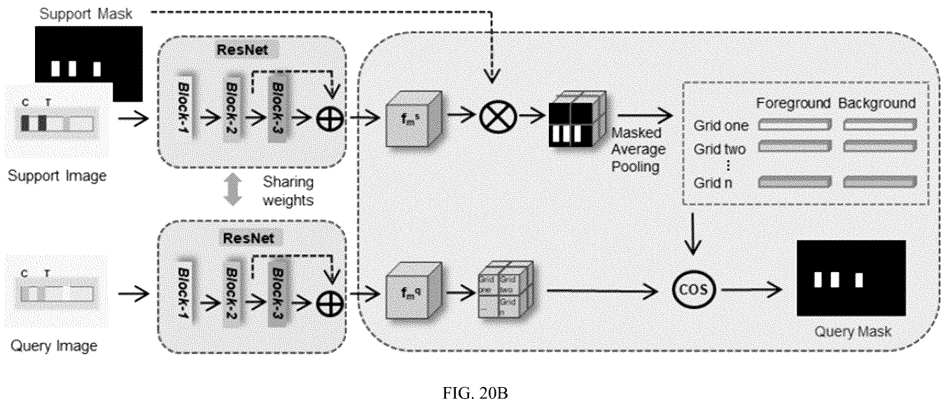

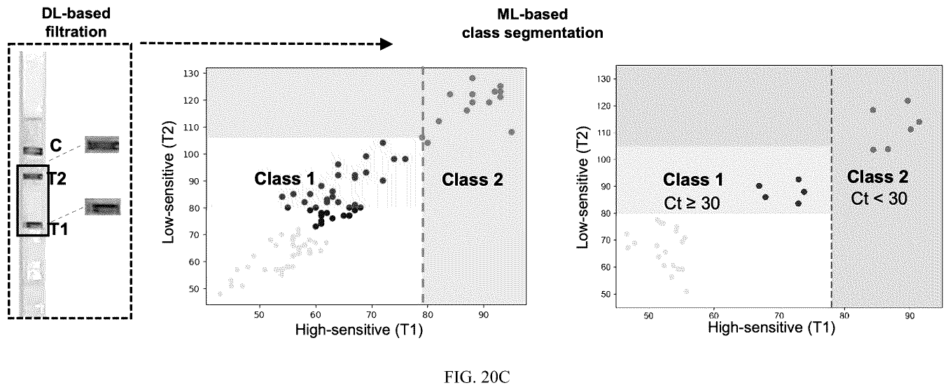

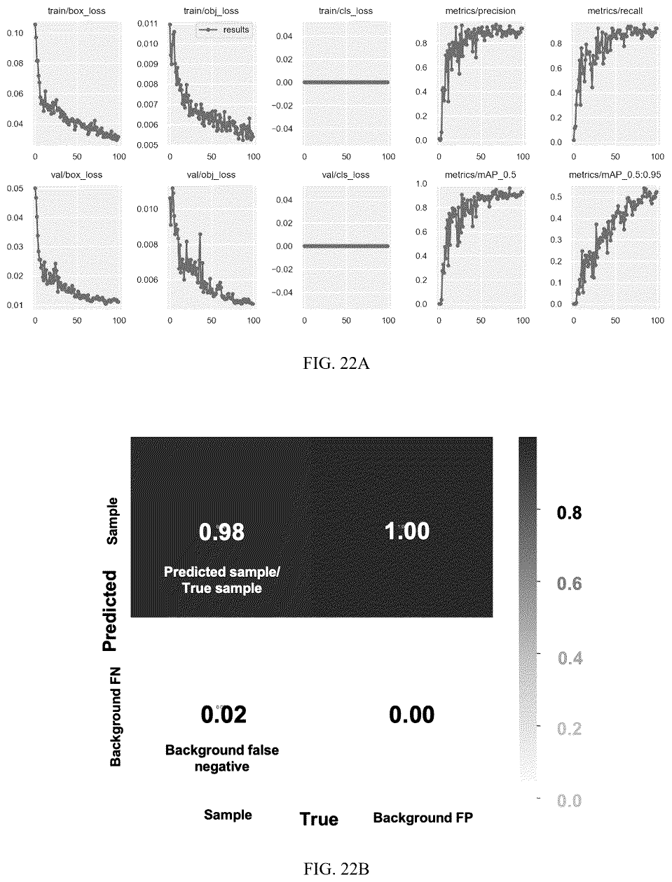

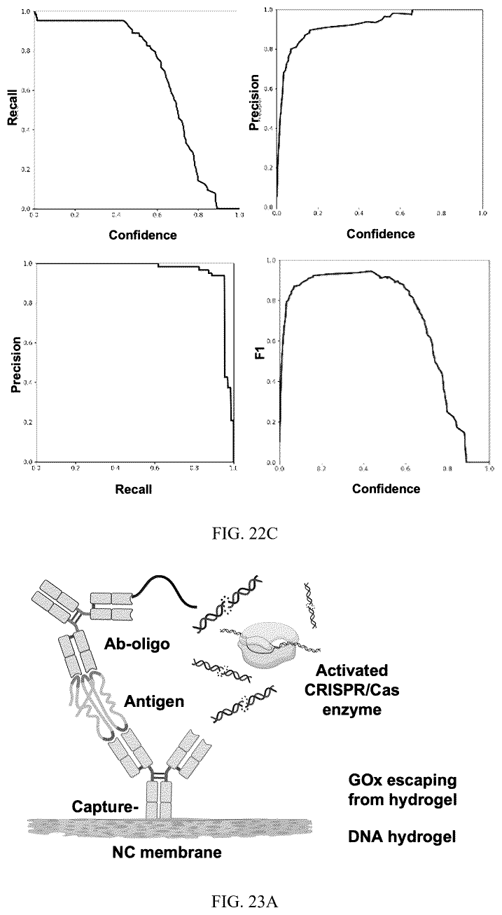

Employing a machine-learning-assisted Band Results Readout System (BRRS) to distinguish chromogenic bands from complex backgrounds and obtain more reliable diagnostic results (FIG. 20A), it is possible to accurately determine high viral load samples from the rest via a threshold determined by the two test bands (10³ cps/μL for HPV tests, Ct=30 for the respiratory pathogens). The BRRS consisted of three steps: imaging the whole strip using a smartphone camera within a black chamber, isolating the test bands in the strip with a deep-learning-based segmentation model, and classifying the results using machine-learning-based classification models. Specifically, the initial photos were first segmented into the same size with single color band using YOLOv5 (FIG. 20B), a widely used deep-learning model which facilitated precise extraction of image features⁴³ i.e., the test bands on the CoRPLA strip (FIGS. 21, 22A, 22B, and 22C). We trained the YOLOv5 model with our own manually circled out bounding boxes as the ground truth on our CoRPLA strip and the trained model can classify the HPV positive or negative from the class probability outputs with a 98% sensitivity. The average RGB values of each segmented band were extracted from ImageJ⁴⁴ for further semi-quantitative analysis. The RGB value of the segmented band was first classified by the random forest model 45. Subsequently, an agglomerative clustering classification model⁴⁶ was used to assign the two viral concentrations (L1, L2) to each band (FIG. 20C). The agglomerative clustering method performed best of the clustering methods we tested, especially in the high-sensitive range.

### Example 10—Detection of Other Biomarkers Like Antigen Using CORPLA

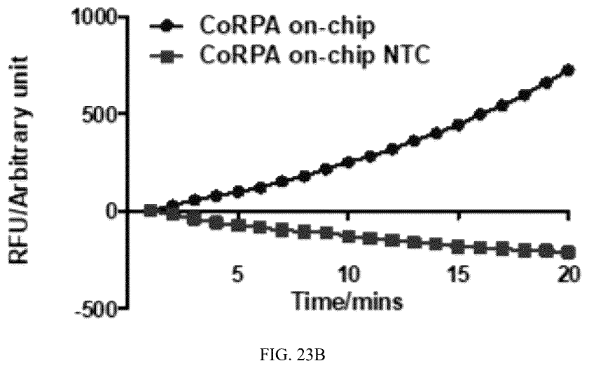

By adding an activator sequence for gRNA recognition onto the reporter antibody (FIG. 23A), CoRPLA can help to detect other biomarkers using specific antigens with sensitivity as high as 1 nM (FIG. 23B).

### Example 11—Detection of Interstitial Field Sample by Micro-Needle Patch Using CORPLA

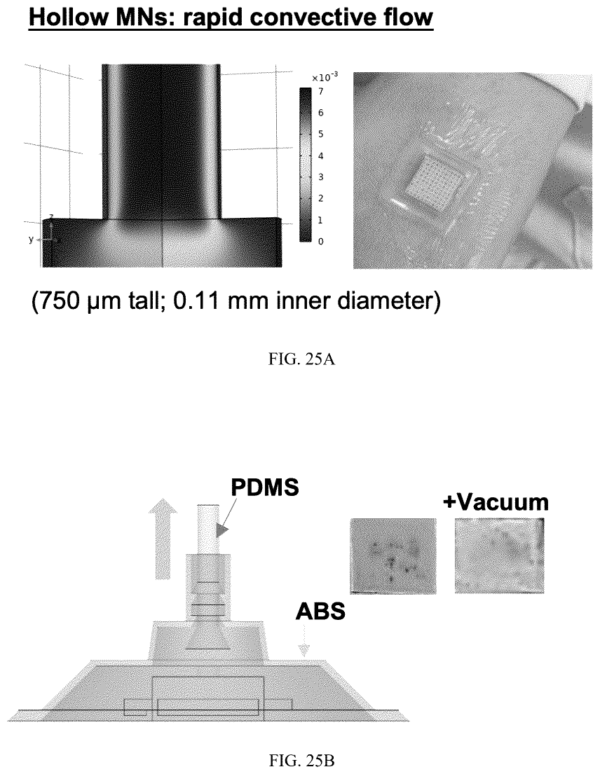

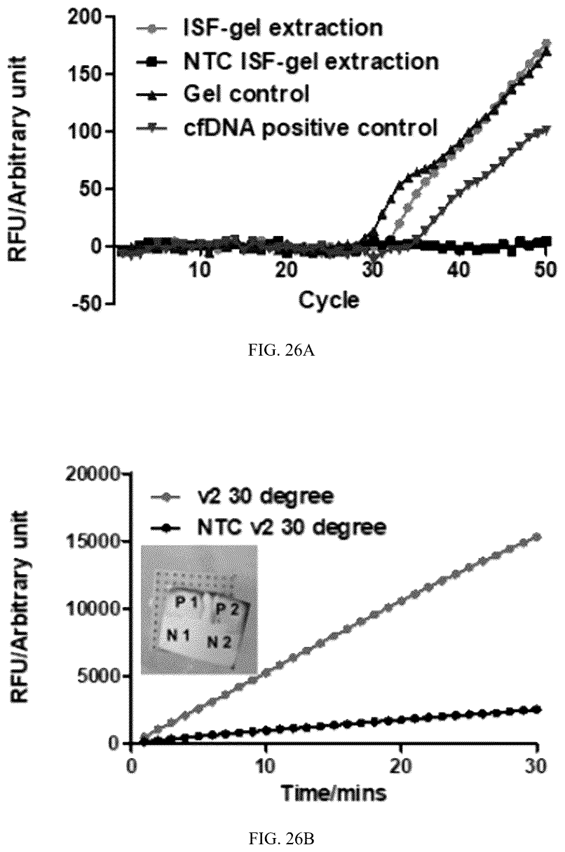

To expand the diversity of sampling methods for wearable biosensors, our testing strip is compatible with a micro-needle patch benefiting from a vacuum valve with suction force on skin surface (FIGS. 25A and 25B), thus overcoming the sample collection barrier and meeting various monitoring needs in clinical patients. The use of a microneedle format resulted in a square patch test paper, leading to a 4-fold signal inhibition in the CoRPLA test due to irregular and limited sampling points, despite the agarose gel not affecting amplification efficiency (FIGS. 26A and 26B).

### EXEMPLARY EMBODIMENTS

Embodiment 1. A method of amplicon-depleted CRISPR/Cas-regulated isothermal molecular assay at skin temperature enabling multi-level, equipment-free, home-based self-testing of viral infections, the method comprising:
a) obtaining a sample containing a target nucleic acid sequence containing target region for primer binding;
b) hybridizing a forward RPLA primer to a target region, wherein the forward RPLA primer comprises a complementary sequence to the target region and a PAM code sequence downstream of the complementary sequence;
c) hybridizing a backward RPLA primer to the primer target region of the displaced sequence;
d) extending the backward RPLA primer to yield a dumbbell sequence containing a loop structure;
e) extending the forward and backward RPLA primer to yield a displaced sequence in the loop containing amplicon, wherein the displaced sequence contains the sgRNA binding region;
f) amplifying the dumbbell sequence by CRISPR/Cas activation that specifically recognizes the sgRNA binding regions using cis cleavage activity; and
g) cleaving the surrounding reporters by collateral cleavage activity right after activation.

Embodiment 2. The method of embodiment 1, wherein the PAM code sequence is immediately downstream of the complementary sequence or at least 1, 2, 3, 4, 5, 6, 7, 8, 9, or 10 nucleotides downstream of the complementary sequence.

Embodiment 3. The method of any preceding embodiments, wherein the forward RPLA primer further comprises a primer binding region.

Embodiment 4. The method of any preceding embodiments, wherein the sample is whole blood, plasma, serum, lymph, urine, saliva, tears, nasopharyngeal secretions, interstitial field, or any combination thereof.

Embodiment 5. The method of any preceding embodiments, wherein amplifying the dumbbell sequence uses a Bsu DNA polymerase under a low temperature range from 28 to 42 degrees.

Embodiment 6. The method of any preceding embodiments, wherein the fully extended amplicon sequence after forming the dumbbell structure contains a sgRNA binding sequence for single guide RNA (sgRNA) recognition for CRISPR/Cas12a nucleic acid cleavage.

Embodiment 7. The method of embodiment 6, further comprising:
h) contacting a CRISPR/Cas sgRNA complementary to the amplified dumbbell sequence with reporters, wherein the CRISPR/Cas sgRNA comprises a target binding sequence specific to the primer binding region of the fully extended amplicon after the dumbbell structure.

Embodiment 8. The method of embodiment 6, wherein the sgRNA targets a sequence adjacent to or at least 1, 2, 3, 4, 5, 6, 7, 8, 9, or 10 nucleotides upstream or downstream of the PAM code sequence.

Embodiment 9. The method of embodiment 6, wherein the sample contains a pathogen with genetic materials.

Embodiment 10. The method of any preceding embodiments, wherein the target nucleic acid sequence is single-stranded RNA, single-stranded DNA, or double-stranded DNA.

Embodiment 11. The method of any preceding embodiments, further comprising adding 7.5% glycine to the CRISPR/Cas12a solution to isolate it from RPLA reagents in one-pot reaction.

Embodiment 12. The method of any preceding embodiments, wherein the reporter is a single-stranded or a double-stranded DNA fluorescent reporter.

Embodiment 13. The method of any preceding embodiments, wherein the single-stranded or a double-stranded DNA fluorescent reporter is labeled at the 3′ end with a fluorophore and at the 5′ end with a quencher and further comprises a TTTTT sequence between the fluorophore and the quencher.

Embodiment 14. The method of any preceding embodiments, wherein the reporter is a single-stranded or a double-stranded DNA electrochemical reporter.

Embodiment 15. The method of any preceding embodiments, wherein the single-stranded or a double-stranded DNA electrochemical reporter is labeled at the 3′ end and 5′ end with a methylene blue label and further comprises a 30 to 60 bases-TTTTT-sequence between the two labels.

Embodiment 16. The method of embodiment 7, wherein the reporter is a dsDNA scaffold within a DNA hydrogel.

Embodiment 17. The method of embodiment 7, further comprising cleaving the reporter to generate a transduced signal from excitation of the cleaved reporter after CRISPR/Cas activation.

Embodiment 18. The method of embodiment 17, further comprising detecting a colorimetric signal using a paper-based testing strip detection with the reporter labelled with a colorimetric initiator.

Embodiment 19. The method of embodiment 18, wherein the colorimetric initiator is glucose oxidase.

Embodiment 20. The method of embodiment 18, further comprising a colorimetric cascade including reagents glucose, horseradish peroxidase (HRP), and the chromogenic mixture of sodium 3,5-dichloro-2-hydroxy-benzenesulphonic acid (DHBS) and 4-aminoantipyrine (4-AAP) on the test bands of the testing strip.

Embodiment 21. The method of embodiment 20, wherein the amounts of the sodium 3,5-dichloro-2-hydroxybenzenesulfonate (DHBS) substrate is adjustable for tunable sensitivities for multi-level visual readout.

Embodiment 22. The method of embodiment 18, further comprising a mobile phone-assisted multi-level diagnostic Band Results Readout System (BRRS) using machine learning interpretation.

Embodiment 23. The method of embodiment 22, wherein the BRRS is realized by Yolov5s to recognize the test band region so that the initial photos were first segmented into the same size for further analysis and viral load classification.

Embodiment 24. The method of embodiment 18, further comprising the activator modified onto an antibody-based reporter make the reaction compatible with the antibody-based detection utilizing the transduced signal from excitation of the cleaved reporter after CRISPR/Cas activation.

Embodiment 25. The method of embodiment 18, further comprising other sampling method such as the micro-needle extraction of interstitial field (ISF).

### REFERENCES

1. Zhong, N. S. et al. Epidemiology and cause of severe acute respiratory syndrome (SARS) in Guangdong, People's Republic of China, in February 2003. The Lancet 362, 1353-1358 (2003).
2. Musso, D., Ko, A. I. & Baud, D. Zika Virus Infection—After the Pandemic. New England Journal of Medicine 381, 1444-1457 (2019).
3. Wang, C., Horby, P. W., Hayden, F. G. & Gao, G. F. A novel coronavirus outbreak of global health concern. The Lancet 395, 470-473 (2020).
4. Dinnes, J. et al. Rapid, point-of-care antigen and molecular-based tests for diagnosis of SARS-CoV-2 infection. Cochrane Database of Systematic Reviews 2020, (2020).
5. Peeling, R. W. et al. Serology testing in the COVID-19 pandemic response. Lancet Infect Dis 20, e245-e249 (2020).
6. Long, Q. X. et al. Antibody responses to SARS-CoV-2 in patients with COVID-19. Nature Medicine 2020 26:6 26, 845-848 (2020).
7. Balmaseda, A. et al. Antibody-based assay discriminates Zika virus infection from other flaviviruses. Proc Natl Acad Sci USA 114, 8384-8389 (2017).
8. Huang, C. et al. Clinical features of patients infected with 2019 novel coronavirus in Wuhan, China. The Lancet 395, 497-506 (2020).
9. Faye, O. et al. One-step RT-PCR for detection of Zika virus. Journal of Clinical Virology 43, 96-101 (2008).
10. Wang, W. et al. Detection of SARS-CoV-2 in Different Types of Clinical Specimens. JAMA 323, 1843-1844 (2020).
11. Myhrvold, C. et al. Field-deployable viral diagnostics using CRISPR-Cas13. Science (1979) 360, 444-448 (2018).
12. Kang, T., Lu, J., Yu, T., Long, Y. & Liu, G. Advances in nucleic acid amplification techniques (NAATs): COVID-19 point-of-care diagnostics as an example. Biosens Bioelectron 206, 114109 (2022).
13. Broughton, J. P. et al. CRISPR-Cas12-based detection of SARS-CoV-2. Nat Biotechnol (2020) doi: 10.1038/s41587-020-0513-4.
14. Joung, J. et al. Detection of SARS-CoV-2 with SHERLOCK One-Pot Testing. New England Journal of Medicine 383, 1492-1494 (2020).
15. Ackerman, C. M. et al. Massively multiplexed nucleic acid detection with Cas13. Nature (2020) doi: 10.1038/s41586-020-2279-8.
16. Broughton, J. P. et al. CRISPR-Cas12-based detection of SARS-CoV-2. Nature Biotechnology 2020 38:7 38, 870-874 (2020).
17. Chandrasekaran, S. S. et al. Rapid detection of SARS-CoV-2 RNA in saliva via Cas13. Nature Biomedical Engineering 2022 6:8 6, 944-956 (2022).
18. Hardinge, P. & Murray, J. A. H. Reduced False Positives and Improved Reporting of Loop-Mediated Isothermal Amplification using Quenched Fluorescent Primers. Scientific Reports 2019 9:19, 1-13 (2019).
19. Ackerman, C. M. et al. Massively multiplexed nucleic acid detection with Cas13. Nature (2020) doi: 10.1038/s41586-020-2279-8.
20. Lee Yu, H. L., Zhang, Y. & Hsing, I. M. Kinetically-enhanced DNA detection via multiple-pass exonuclease III-aided target recycling. Analyst 142, 4782-4787 (2017).
21. Deng, F. et al. Topological barrier to Cas12a activation by circular DNA nanostructures facilitates autocatalysis and transforms DNA/RNA sensing. Nature Communications 2024 15:1 15, 1-16 (2024).
22. Lu, S. et al. Fast and sensitive detection of SARS-CoV-2 RNA using suboptimal protospacer adjacent motifs for Cas12a. Nature Biomedical Engineering 2022 6:3 6, 286-297 (2022).
23. Cao, Y. et al. CoLAMP: CRISPR-based one-pot loop-mediated isothermal amplification enables at-home diagnosis of SARS-CoV-2 RNA with nearly eliminated contamination utilizing amplicons depletion strategy. Biosens Bioelectron 236, 115402 (2023).
24. Bao, Y. et al. CUT-LAMP: Contamination-Free Loop-Mediated Isothermal Amplification Based on the CRISPR/Cas9 Cleavage. ACS Sens 5, 1082-1091 (2020).
25. Zadeh, J. N. et al. Software News and Updates NUPACK: Analysis and Design of Nucleic Acid Systems. J Comput Chem 32, 170-173 (2011).
26. Yates, L. A. et al. A structural and dynamic model for the assembly of Replication Protein A on single-stranded DNA. Nature Communications 2018 9:19, 1-14 (2018).
27. Spegg, V. et al. Phase separation properties of RPA combine high-affinity ssDNA binding with dynamic condensate functions at telomeres. Nature Structural & Molecular Biology 2023 30:4 30, 451-462 (2023).
28. Li, Y., Fan, P., Zhou, S. & Zhang, L. Loop-mediated isothermal amplification (LAMP): A novel rapid detection platform for pathogens. Microb Pathog 107, 54-61 (2017).
29. Carvalho, F., Magalhaes, C., Fernandez-Llimos, F., Mendes, J. & Gonçalves, J. Skin temperature response to thermal stimulus in patients with hyperhidrosis: A comparative study. J Therm Biol 109, 103322 (2022).
30 Nguyen, L. T., Smith, B. M. & Jain, P. K. Enhancement of trans-cleavage activity of Cas12a with engineered crRNA enables amplified nucleic acid detection. Nature Communications 2020 11:1 11, 1-13 (2020).
31. Harrington, L. B. et al. CRISPR-Cas12a target binding unleashes indiscriminate single-stranded DNase activity. Science (1979) 360, 436-439 (2018).
32. Raghunathan, S., Kozlov, A. G., Lohman, T. M. & Waksman, G. Structure of the DNA binding domain of E. coli SSB bound to ssDNA. Nature Structural Biology 2000 7:8 7, 648-652 (2000).
33. Lobato, I. M. & O'Sullivan, C. K. Recombinase polymerase amplification: Basics, applications and recent advances. TrAC—Trends in Analytical Chemistry 98, 19-35 (2018).
34. Lee Yu, H., Cao, Y., Lu, X. & Hsing, I. M. Detection of rare variant alleles using the AsCas12a double-stranded DNA trans-cleavage activity. Biosens Bioelectron 189, 113382 (2021).
35. Fonfara, I., Richter, H., BratoviA, M., Le Rhun, A. & Charpentier, E. The CRISPR-associated DNA-cleaving enzyme Cpf1 also processes precursor CRISPR RNA. Nature (2016) doi: 10.1038/nature17945.
36. Siddiqui, K. S. & Cavicchioli, R. Cold-adapted enzymes. Annu Rev Biochem 75, 403-433 (2006).
37. Feng, W., Zhang, H. & Le, X. C. Signal Amplification by the trans-Cleavage Activity of CRISPR-Cas Systems: Kinetics and Performance. Anal Chem 95, 206-217 (2023).
38. Nalefski, E. A. et al. Kinetic analysis of Cas12a and Cas13a RNA-Guided nucleases for development of improved CRISPR-Based diagnostics. iScience 24, 102996 (2021).
39. Valloly, P. & Roy, R. Nucleic Acid Quantification with Amplicon Yield in Recombinase Polymerase Amplification. Anal Chem 94, 13897-13905 (2022).
40. Ganguli, A. et al. Rapid isothermal amplification and portable detection system for SARS-CoV-2. Proc Natl Acad Sci USA 117, 22727-22735 (2020).
41. Xing, Y. et al. Self-assembled DNA hydrogels with designable thermal and enzymatic responsiveness. liuds.chem.tsinghua.edu.cnY Xing, E Cheng, Y Yang, P Chen, T Zhang, Y Sun, Z Yang, D LiuAdvanced Materials, 2011·liuds.chem.tsinghua.edu.cn 23, 1117-1121 (2011).
42. Bi, Y. et al. Smart Bilayer Polyacrylamide/DNA Hybrid Hydrogel Film Actuators Exhibiting Programmable Responsive and Reversible Macroscopic Shape Deformations. Small 16, 1906998 (2020).
43. Wu, W. et al. Application of local fully Convolutional Neural Network combined with YOLO v5 algorithm in small target detection of remote sensing image. PLoS One 16, e0259283 (2021).
44. Schneider, C. A., Rasband, W. S. & Eliceiri, K. W. NIH Image to ImageJ: 25 years of image analysis. Nature Methods 2012 9:79, 671-675 (2012).
45. Breiman, L. Random forests. Mach Learn 45, 5-32 (2001).
46. Müllner, D. Modern hierarchical, agglomerative clustering algorithms. (2011).

We claim:

1. A method of amplicon-depleted CRISPR/Cas-regulated isothermal molecular assay at skin temperature enabling multi-level, equipment-free, home-based self-testing of viral infections, the method comprising:
a) obtaining a sample comprising a target nucleic acid sequence containing target region for primer binding;
b) hybridizing a forward Recombinase Polymerase Loop-mediated Amplification (RPLA) primer to the target region, wherein the forward RPLA primer comprises a complementary sequence to the target region and a protospacer adjacent motif (PAM) code sequence downstream of the complementary sequence;
c) hybridizing a backward RPLA primer to the primer target region of the displaced sequence;
d) extending the backward RPLA primer to yield a dumbbell sequence containing a loop structure;
e) extending the forward and backward RPLA primers to yield a displaced sequence in the loop containing amplicon, wherein the displaced sequence contains the sgRNA binding region;
f) amplifying the dumbbell sequence by CRISPR/Cas activation that specifically recognizes single guide RNA (sgRNA) binding regions using cis cleavage activity; and
g) cleaving the surrounding reporters by collateral cleavage activity right after activation.

2. The method of claim 1, wherein the PAM code sequence is immediately downstream of the complementary sequence or at least 1, 2, 3, 4, 5, 6, 7, 8, 9, or 10 nucleotides downstream of the complementary sequence.

3. The method of claim 1, wherein the forward RPLA primer further comprises a primer binding region.

4. The method of claim 1, wherein the sample is whole blood, plasma, serum, lymph, urine, saliva, tears, nasopharyngeal secretions, interstitial field, or any combination thereof.

5. The method of claim 1, wherein amplifying the dumbbell sequence uses a Bsu DNA polymerase under a low temperature range from 28 to 42 degrees.

6. The method of claim 1, wherein the fully extended amplicon sequence after forming the dumbbell structure contains a sgRNA binding sequence for sgRNA recognition for CRISPR/Cas12a nucleic acid cleavage.

7. The method of claim 6, further comprising: contacting a CRISPR/Cas sgRNA complementary to the amplified dumbbell sequence with reporters, wherein the CRISPR/Cas sgRNA comprises a target binding sequence specific to the primer binding region of the fully extended amplicon after the dumbbell structure.

8. The method of claim 6, wherein the sgRNA targets a sequence adjacent to or at least 1, 2, 3, 4, 5, 6, 7, 8, 9, or 10 nucleotides upstream or downstream of the PAM code sequence.

9. The method of claim 1, wherein the sample contains a pathogen with genetic material.

10. The method of claim 1, wherein the target nucleic acid sequence is single-stranded RNA, single-stranded DNA, or double-stranded DNA.

11. The method of claim 1, further comprising adding 7.5% glycine to the CRISPR/Cas12a solution to isolate it from RPLA reagents in one-pot reaction.

12. The method of claim 7, wherein the reporter is a single-stranded or a double-stranded DNA fluorescent reporter.

13. The method of claim 12, wherein the single-stranded or a double-stranded DNA fluorescent reporter is labeled at the 3′ end with a fluorophore and at the 5′ end with a quencher and further comprises a TTTTT sequence between the fluorophore and the quencher.

14. The method of claim 7, wherein the reporter is a single-stranded or a double-stranded DNA electrochemical reporter.

15. The method of claim 14, wherein the single-stranded or a double-stranded DNA electrochemical reporter is labeled at its 3′ end and 5′ end with a methylene blue label and further comprises a 30 to 60 bases-TTTTT-sequence between the two labels.

16. The method of claim 7, wherein the reporter is a dsDNA scaffold within a DNA hydrogel.

17. The method of claim 7, further comprising cleaving the reporter to generate a transduced signal from excitation of the cleaved reporter after CRISPR/Cas activation.

18. The method of claim 17, further comprising detecting a colorimetric signal using a paper-based testing strip detection with the reporter labelled with a colorimetric initiator.

19. The method of claim 18, wherein the colorimetric initiator is glucose oxidase.

20. The method of claim 18, further comprising a colorimetric cascade including reagents glucose, horseradish peroxidase (HRP), and the chromogenic mixture of sodium 3,5-dichloro-2-hydroxy-benzenesulphonic acid (DHBS) and 4-aminoantipyrine (4-AAP) on the test bands of the testing strip.

21. The method of claim 20, wherein the amount of the sodium 3,5-dichloro-2-hydroxybenzenesulfonate (DHBS) substrate is adjustable for tunable sensitivities for multi-level visual readout.

22. The method of claim 18, further comprising a mobile phone-assisted multi-level diagnostic Band Results Readout System (BRRS) using machine learning interpretation.

23. The method of claim 22, wherein the BRRS is realized by Yolov5s to recognize the test band region so that initial photos are first segmented into a same size for further analysis and viral load classification.

24. The method of claim 18, further comprising the activator modified onto an antibody-based reporter make the reaction compatible with the antibody-based detection utilizing the transduced signal from excitation of the cleaved reporter after CRISPR/Cas activation.

25. The method of claim 18, further comprising another sampling method such as micro-needle extraction of interstitial field (ISF).
